(19) United States
(12) Patent Application Publication
(10) Pub. No.: US 2013/0123117 A1
(43) Pub. Date: May 16, 2013

(54) CAPTURE PROBE AND ASSAY FOR ANALYSIS OF FRAGMENTED NUCLEIC ACIDS

(71) Applicant: The Board of Trustees of the Leland Stanford Junio, Palo Alto, CA (US)

(72) Inventors: Hua Xu, Sunnyvale, CA (US); Georges Natsoulis, Kensington, CA (US); Hanlee P. Ji, Stanford, CA (US)

(73) Assignee: The Board of Trustees of the Leland Stanford Junior University, Palo Alto, CA (US)

(21) Appl. No.: 13/678,355

(22) Filed: Nov. 15, 2012

Related U.S. Application Data
(60) Provisional application No. 61/560,412, filed on Nov. 16, 2011.

(51) Int. Cl. C12Q 1/68

(52) U.S. Cl. CPC C12Q 1/6876
USPC 506/2; 506/16

(57) ABSTRACT

Disclosed is an efficient and scalable method for targeted resequencing and variant identification of nucleic acids such as genomic DNA found in single stranded, fragmented form, such as in a clinical sample of formalin-fixed, paraffin-embedded (FFPE) tissue. The method uses a large number of capture probes mixed with the sample in the presence of a 5′ to 3′ exonuclease, a 3′ to 5′ exonuclease, a ligase, and a universal amplification oligonucleotide that hybridizes to the various capture probes. The nucleases act on ssDNA, not dsDNA. A single stranded circle is formed by the ligase, and is then amplified to produce a population (library) of double stranded linear DNA molecules that are suitable for sequencing. It is shown that the library produces a high degree of fidelity to the original sample, and predictable base changes are shown.

### CROSS-REFERENCE TO RELATED APPLICATIONS

This application claims priority from U.S. Provisional Patent Application No. 61/560,412 filed on Nov. 16, 2011, which is hereby incorporated by reference in its entirety.

### STATEMENT OF GOVERNMENTAL SUPPORT

This invention was made with government support under contracts 2P01HG000205 and R21CA 140089-01A1 awarded by the National Institutes of Health. The government has certain rights in this invention.

### REFERENCE TO SEQUENCE LISTING, COMPUTER PROGRAM, OR COMPACT DISK

The instant application contains a Sequence Listing which has been submitted in ASCII format via EFS-Web and is hereby incorporated by reference in its entirety. Said ASCII copy, created on Oct. 26, 2012, is named 381596US.txt and is 828,319 bytes in size.

### BACKGROUND OF THE INVENTION

1. Field of the Invention

The present invention relates to the field of nucleic analysis, and, more particularly, to methods for contacting fragmented nucleic acids, such as genomic DNA with probes and enzymes whereby selected portions of the genomic DNA are amplified and assayed.

2. Related Art

Presented below is background information on certain aspects of the present invention as they may relate to technical features referred to in the detailed description, but not necessarily described in detail. That is, individual parts or methods used in the present invention may be described in greater detail in the materials discussed below, which materials may provide further guidance to those skilled in the art for making or using certain aspects of the present invention as claimed. The discussion below should not be construed as an admission as to the relevance of the information to any claims herein or the prior art effect of the material described.

Next generation DNA sequencing (NGS) has revolutionized genetics by enabling one to routinely sequence human genomes, either in their entirety or specific subsets. While NGS advances have dramatically increased our ability to identify disease-related genetic variants, the widespread application of NGS-based approaches to clinical populations faces some limitations. Citing an example, NGS-based discovery of cancer mutations for large translational and clinical studies is severely restricted by the availability of clinical samples from which one can extract high quality genomic DNA. The vast majority of cancers samples like gastric and colorectal cancer are processed with formalin fixed paraffin embedding (FFPE) of tissues. For clinical pathology laboratories, this is a preservation method because (1) it maintains morphological features of the tumor, (2) enables histopathologic examination with a number of staining processes and (3) can be stored indefinitely at room temperature. However, the fixation process causes irreversible damage to the sample genomic DNA via cross linkages and increased fragmentation. As a result, genomic DNA extracted from FFPE material is often of poor quality. Furthermore, FFPE-extracted genomic DNA is generally in a single stranded form because of the need for high temperature incubations to melt the paraffin. Therefore, the analysis of FFPE-derived genomic DNA using PCR-based assays is difficult. Overall, these issues restrict our ability to conduct clinical population genetic studies and genetic diagnostic development using these valuable samples.

A variety of methods have been developed to enrich specific regions of the human genome. These include in-solution hybridization enrichment, multiplexed-PCR and targeted circularization approaches. Hybrid selection methods apply immobilized oligonucleotides on either microarrays [1-3] or beads [4] to enrich genomic targets from a modified DNA sample. In multiplex-PCR [5], complex primer sets can be utilized to selectively amplify targeted regions prior to modifying DNA for the sequencer. Highly parallel simplex PCR reactions can be conducted with microdroplet technology [6]. In-solution oligonucleotide-based approaches such as molecular inversion probes (MIPs) capture targets by DNA synthesis across the target and ligation that result in circularization of the capture oligonucleotides [7, 8]. Citing another in-solution approach, targeted genomic circularization (TGC) directly captures a genomic DNA target by converting it into a target specific circle using in-solution capture oligonucleotides [9].

There are limitations with all of the previously described capture methods on genomic DNA from FFPE samples. For example, hybridization enrichment has been applied to cancer samples for single nucleotide variation (SNV) detection [10]. For example, Kerick et al. used the Agilent in-solution hybridization method to investigate reproducibility of SNV detection comparing genomic DNA from FFPE to flash-frozen samples. They demonstrated a false positive rate of approximately 1% when using sequencing coverage greater than 20× coverage. This translates into 1 false mutation caller for every 100 variants identified. In addition, hybridization-based methods have high levels of off-target capture, involve complex workflows that require additional PCR amplification and sample preparation steps. MIP technology has potential advantages for degraded genomic DNA from FFPE samples, but the capture reaction is inefficient for larger targets beyond 200 bps and the assay is extremely complicated in its implementation [11]. Furthermore, with MIPs, the captured regions contain 20 bps of the oligonucleotide-derived sequences and the rest is the reverse-complement of the template DNA, not the original DNA strand. This requires some degree of bioinformatic processing to eliminate synthetic sequence. Capture with the targeted genomic circularization relies on the presence of existing restriction sites in double stranded DNA and requires multiple restriction enzymes which increase the number of reactions needed for a given sample [9]. This can limit the efficiency of capture coverage due to the absence of a suitable restriction site. Furthermore, TGC-capture requires double stranded DNA for restriction enzyme fragmentation while FFPE-derived genomic DNA is generally single stranded. Whole genome amplification using random primers followed by an end-repair step can be used to sequence FFPE-derived genomic DNA, but these amplification steps can skew the representation of certain region even before the capture reaction.

### Specific Patents and Publications

Dahl et al., “Multiplex amplification enabled by selective circularization of large sets of genomic DNA fragments,” Nucleic Acids Res. 33 e71 (2005), discloses a method for multiplex amplification which uses a general primer pair motif and a vector oligonucleotide selector probe, where the circularization procedure starts with digestion of the DNA to generate targets.

US patent publication 2008/0199916, by Zheng et al., published Aug. 21, 2008, entitled “Multiplex targeted amplification using flap nuclease,” discloses the use of UDG (uracil-DNA glycosylase) and a flap exonuclease.

PG Pub 2007/0128635 by Macevicz, entitled “Selected Amplification of Polynucleotides,” discloses a method in which fragments and selection oligonucleotides are combined in a reaction mixture comprising the following enzymatic activities: (i) a 5′ flap endonuclease activity, (ii) a DNA polymerase lacking strand displacement activity, (iii) a 3′ single stranded exonuclease activity, and (iv) a ligase activity.

WO 2008/033442 A2, “Methods And Compositions For Performing Low Background Multiplex Nucleic Acid Amplification Reactions,” by Fredriksson et al., discloses a method of amplifying target nucleic acids involving circularizing target amplicons in an amplified composition; and selecting for said circularized target amplicons in said amplified composition.

### BRIEF SUMMARY OF THE INVENTION

The following brief summary is not intended to include all features and aspects of the present invention, nor does it imply that the invention must include all features and aspects discussed in this summary.

The present invention comprises, in certain aspects, methods and materials for detection and analysis of a large number of random fragments of DNA in a sample. The methods can be used for targeted resequencing of DNA. In certain aspects, the present methods employ a mixture of single-stranded polynucleotide capture probes, a number of universal single stranded oligonucleotides (second polynucleotides) each having the same sequence and hybridizing to a portion of the various capture probes; and a mixture comprising exonucleases and a ligase.

In certain aspects, the present invention comprises a composition in the form of a reaction mixture useful for preparing a population of double stranded DNA molecules from a sample containing single stranded polynucleic acids, comprising, preferably in a suitable buffer: (a) a plurality of single stranded capture probes, each capture probe containing (i) 5′ and 3′ end capture arms complementary to specific portions of a polynucleic acid in the sample and (ii) an invariant sequence between the capture arms, whereby a circular structure comprising a specific capture probe and a polynucleic acid sample molecule having regions complementary to the capture arms is formed in the buffer; (b) a plurality of second (“universal”) single stranded polynucleotides having a sequence complementary to the invariant sequence and having amplification sites for amplification of a polynucleic acid in a circular structure; and (c) a 5′ exonuclease, a 3′ exonuclease, and a ligase. While “each” capture probe will contain the defined features, it is not to be implied that “every” capture probe in a composition must have these features.

The single stranded polynucleic acids in the composition may comprise random fragments of human genomic DNA. The fragments may be fixed by crosslinking and embedded in a wax, which makes the composition well suited for dealing with degraded DNA from FFPE samples.

The composition also comprises at least one of amplification primers and a polymerase for amplification. The amplification sites of the composition comprise PCR primer sites, which may be spaced on the universal polynucleotides about 120 to 250 bases apart.

In certain embodiments, the composition (reaction mixture) comprises capture probes having a three part construction: two capture arms on the flanks which are able to capture specific single-stranded genomic DNA and a sequence between the two capture arms which is termed a “universal” sequence in that it is essentially the same (“invariant”) among the different probes. The capture probes may be present in the composition as a set of at least 500 different probes, at least 600 different probes, at least 700 different probes, or at least 1000 different probes, each probe having capture arms complementary to different portions of a single stranded polynucleic acid in the sample and having the same universal probe sequence between the two capture arms.

In certain aspects, the present invention also comprises a method for analyzing single stranded polynucleotides from a sample, comprising the steps of: (a) adding to the sample a plurality of capture probes, each capture probe containing capture arms designed to be complementary to specific portions of a polynucleic acid in the sample and a universal probe sequence between the arms, whereby a circular structure comprising a specific capture probe and a polynucleic acid sample molecule is formed in the buffer; (b) adding to the sample a plurality of universal polynucleotides having a sequence complementary to the universal probe sequence and having amplification sites for amplification of a polynucleic acid in a circular structure; and (c) adding to the sample containing capture probes and universal polynucleotides a mixture of a 5′ exonuclease, a 3′ exonuclease, and a ligase under conditions whereby exonucleases remove bases from the single stranded polynucleotides to form a new 5′ end thereof and a new 3′ end thereof, and the ligase ligates the new 5′ end to the new 3′ end.

The composition and method described above may also comprise a 5′ exonuclease, which may be Exonuclease I; a 3′ exonuclease, which may be a polymerase or a thermostable polymerase; and a ligase, which may be a thermostable DNA ligase. As described below, the capture arms may hybridize to various portions of the DNA in the sample, leaving “flaps”, which are removed by the exonucleases.

In certain aspects, the present invention further contemplates a method for analyzing single stranded polynucleotides from a sample, comprising the steps of: (a) adding to the sample a plurality of capture probes, each capture probe containing capture arms complementary to specific portions of a polynucleic acid in the sample and a universal probe sequence between the arms, whereby a circular structure comprising a specific capture probe and a polynucleic acid sample molecule is formed in the buffer; (b) adding to the sample a plurality of universal polynucleotides having a sequence complementary to the universal probe sequence and having amplification sites for amplification of a polynucleic acid in a circular structure; and (c) adding to the sample containing capture probes and universal polynucleotides a mixture of a 5′ exonuclease, a 3′ exonuclease, and a ligase under conditions whereby exonucleases remove bases from the single stranded polynucleotides to form a new 5′ end thereof and a new 3′ end thereof, and the ligase ligates the new 5′ end to the new 3′ end; (d) adding to the sample a polymerase and polymerase primers; and (e) conducting a polymerase chain reaction using the polymerase primers for amplification of a portion of a single stranded polynucleotide captured by a corresponding capture probe.

The above method may further comprise the step of sequencing amplified polynucleotides from step (e). The polymerase chain reaction conducted step (e) may utilize an annealing temperature of between about 45 degrees Celsius and 55 degrees Celsius.

The analyzing of the single stranded polynucleotides from a sample may comprise analyzing polynucleotides from a preserved tissue sample or analyzing polynucleotides from a preserved tissue sample and analyzing polynucleotides from a fresh sample from the same individual.

In certain aspects, the present invention also comprises the preparation of a composition as described herein using a kit. The kit may comprise a set of capture probes and universal oligos. Other reagents, such as enzymes may also be included in the kit. An exemplary set of 628 capture polynucleotides is described in the accompanying sequence listing.

### BRIEF DESCRIPTION OF THE DRAWINGS

FIG. 1A, 1B is a schematic diagram illustrating an overview of the single stranded DNA capture assay.

FIGS. 2A, 2B, and 2C is a set of graphs showing the sequencing coverage of targeted resequencing on matched FFPE versus flash-frozen genomic DNA sources in exemplary patients 751 (FIG. 2A), patient 761 (FIG. 2B) and patient 780 (FIG. 2C). Coverage exceeded 85% of all captured regions in each case.

FIG. 3 is a scatter plot showing where the 2^{nd} base frequency of a given variant is compared from targeted resequencing of genomic DNA from matched flash-frozen versus FFPE samples. The x-axis represents the 2^{nd} base frequency of SNVs identified from FFPE targeted resequencing compared to the y-axis, which indicates the variant base fraction from the flash-frozen genomic DNA.

### DETAILED DESCRIPTION OF THE PREFERRED EMBODIMENT

### Overview

Described herein is a novel DNA targeting and enrichment method particularly suited for analysis of samples containing fragmented single stranded nucleic acids, such as genomic DNA fragments in a biopsy sample. The method results in highly multiplexed amplification of selected portions of the sample nucleic acid, i.e., the reaction mixture may contain hundreds or thousands of different capture probes for amplification of sample DNA regions spanned by the capture probes. The amplified portions from the reaction may be further analyzed, e.g. by sequencing the amplified portions.

The present method is an improvement of a previously described technique that required double stranded DNA as input and required that the targeting oligonucleotide probes be placed adjacent to certain restriction sites. For the present approach, the hybridization arms of the capture oligonucleotides do not require a restriction site and the input DNA can be single stranded. This improves the flexibility and the coverage of the design. An important feature of the present capture approach involves using single stranded DNA as input material. Given the need for high heat during processing, the majority of formalin fixed and paraffin embedded (FFPE) derived genomic DNA molecules are generally single stranded. The present approach has a major advantage compared to other methods that rely exclusively on enzymatic manipulations of double stranded genomic DNA. The capture performance is comparable when using genomic DNA derived from flash-frozen versus FFPE processed tissue. Eighty five percent of the heterozygote SNV detected from high quality genomic DNA extracted flash-frozen samples were also detected in targeted resequencing data from the matched FFPE samples. The number of false positive FFPE-specific SNV calls are exceptionally low at one per every 12 Kb of targeted genomic sequence.

While multiplexed capture assays for hundreds of genomic regions in the present examples is described herein, it is believed the reaction could be scaled to thousands. As published, efficient capture using pools of 5,000 oligonucleotides for restriction enzyme-based targeted circularization has been achieved and it is believed that this new method will scale similarly. For most of the results presented here, we used 4 indexed samples per lane of sequencing (2 flash frozen and 2 FFPE samples). Targeted resequencing projects involving hundreds of exons in hundreds of FFPE samples are therefore achievable and may be implemented with minimal additional steps in a next generation sequencer such as the Illumina HiSeq or GAIIx. In addition, the application of the present approach is demonstrated using the Illumina MiSeq system which is designed for rapid analysis.

An innovative approach to capture genomic targets from archival genomic DNA with in-solution polynucleotides is described. This approach is fundamentally different than other methods given that it only requires random fragments of single stranded genomic DNA as commonly seen in FFPE samples, is highly scalable for multiplexed target coverage, and does not rely on any whole genome amplification. The capture assay is straightforward, relatively fast and can be implemented with standard molecular biology equipment. The robust performance of the capture assay and comparisons of SNV detection using genomic DNA derived from matched flash-frozen and FFPE samples is demonstrated.

The technology described utilizes oligonucleotide-mediated genomic capture without the need for double stranded template and the reliance on exiting restriction sites. It also alleviates the need to synthesize the complementary stranded of the template DNA, which can result in significant limits such as the target size.

Another novel aspect of this capture process is its ability to add desired sequences (such as the adapter sequences required for cluster generation on the Illumina® sequencing system) to DNA fragments without the need for the multi-step process normally associated with such manipulation. This can greatly simplify and accelerate the construction of sequencing libraries. That is, the original

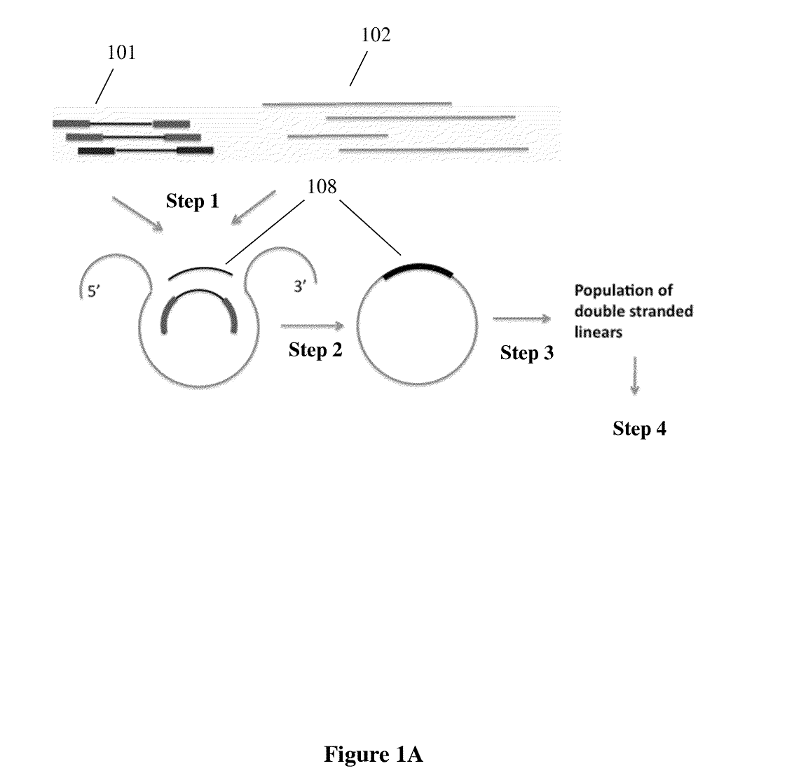

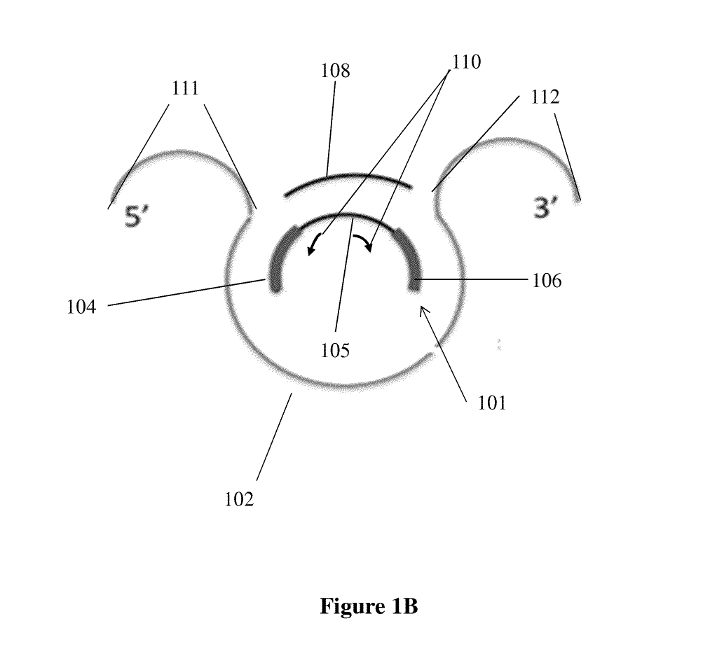

FIGS. 1A and 1B outline the key materials, intermediates and steps of the capture reaction. As shown in these figures, a number of capture probes 101 and a sample containing numerous fragments of single stranded DNA 102 are mixed in a single tube (Step 1). The term “tube” is used for convenience, in that the reaction area could also be a well in a microtiter plate, a chamber in a microfluidic device, etc. The entire reaction occurs in the single tube and this substantially reduces the complexity of the capture assay process. The capture probes and single stranded DNA fragments are mixed in the presence of Ampligase, TaqPol, and ExoI. The capture probes 101 have capture arms that are different in sequence as between capture probes and are complementary to the ends of the portion of sample DNA 102 to be studied.

Denatured single-stranded genomic DNA 102 having a 5′ end and a 3′ end is combined with a pool of polynucleotides, termed “capture probes,” that mediate targeted circularization of the regions of interest. Since the size of DNA 102 is unknown and variable (“random”), portions of the DNA 102 will extend 5′ and 3′ from the hybridization sites, as shown in step 1. The capture probes are single stranded DNA molecules that may be e.g. 80 bases long, or in the range of 40 to 300 bases long. A single capture probe will have 5′ capture arm 104, a middle portion 105 (“universal probe sequence”) and a 3′ capture arm 106 (FIG. 1B). The capture arms 104, 106 are typically on the order of 20 bases long, and have a sequence selected for an individual capture probe to target a pre-determined complementary region on the nucleic acid sample. This complementarity is designed to be 100% complementarity. The region targeted will typically be longer than the capture probe; it may, for example, be an exon of a gene. The middle portion 105 of the capture probe (“universal probe sequence”) is selected to have a sequence that will not hybridize to the nucleic acid sample, and its length is chosen depending on the size of the region of the sample (e.g. genomic DNA) being targeted, and in accordance with the size of the universal oligo. While there are many different capture probe sequences, the middle portion of each capture probe will be essentially the same in each capture probe, in order to hybridize to the universal polynucleotides, as explained below.

Genomic DNA in the sample can come from either flash-frozen or FFPE processed tissue samples. Each capture arm 104, 106 from a single capture probe anneals to a predetermined sequence in a specific genomic DNA fragment 102 containing the complementary sequences. After hybridization, a single-stranded target-specific structure is formed which has 5′ single stranded extension 111 and 3′ single stranded extension 112 of the original genomic target single stranded DNA (FIG. 1B). These extensions 111, 112 of single stranded genomic DNA (that extend past the ends of the targeting arms of the capture probe) are removed or degraded by enzymes. For example 5′ and 3′ extensions (“flaps”) may be removed, respectively, by the 5′ nucleolytic activity of Taq polymerase (activity as disclosed, e.g. in Lyamichev, V., Brow, M. A. & Dahlberg, J. E. (1993) Science 260, 778-783) and the 3′ to 5′ exonucleolytic activity of ExoI [12]. To complete the capture reaction, a universal vector oligonucleotide 108 anneals to the general sequence motif in the middle portion 105 of every capture probe oligonucleotide. Ampligase® thermostable ligase present in the same reaction mix forms covalently closed circles using the universal vector sequence (Step 2). Ampligase® Thermostable DNA Ligase catalyzes NAD-dependent ligation of adjacent 3′-hydroxylated and 5′-phosphorylated termini in duplex DNA structures that are stable at high temperatures.

Once the circle is complete, universal PCR primers 110 can be used to amplify the intervening target genomic DNA fragment, creating a pool of linear amplicons that can be sequenced (Step 3). The primers are oriented, as shown in FIG. 1B, to amplify the target oligonucleotide; they can be amplified either as an intact circle, or after cleavage of the circle. The resulting double stranded linear DNA population that results from amplification of the set of circles created is then submitted to adapter ligation following the standard Illumina library preparation protocol (Step 4). The primers hybridize to sequences within the universal sequences, so that one set of primers may be used to amplify the entire plurality of different capture probe structures.

As shown by arrows 110 in FIG. 1B, the PCR amplification can proceed from the primers through part of the general sequence motif in the middle portion 105 of the capture probe. This allows sequences from this motif to be added to and become part of the 5′ and/or 3′ end of the amplified product. For example, bar codes or ligation adapters can be added by including such sequences in the middle portion 105 of the capture probe. A variety of sequencing methods may be used on the amplified products, including massively parallel methods commercially available from Illumina, Roche 454, Life Technologies, Pacific Biosciences, Helicos, etc. The sequencing aspect of the present methods can be used for SNP analysis as well as SNVs that are associated with disease. The sequencing libraries prepared by the present method can be used for paired-end sequencing to obtain greater information from a ssDNA fragment in the sample.

A variety of buffers can be used with the present compositions. They can contain, e.g. 100 mM Tris-Cl, 500 mM KCl; 600 mM Tris-Cl, 170 mM (NH4)2SO4, 0.1% Tween-20; 375 mM Tris-Cl, 200 mM (NH₄)₂SO₄, 0.1% Tween-20, etc.

### DEFINITIONS

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges:

For conciseness, any range set forth is intended to include any sub-range within the stated range, unless otherwise stated. A sub-range is to be included within a range even though no sub-range is explicitly stated in connection with the range. As a nonlimiting example, a range of 120 to 250 includes a range of 120-121, 120-130, 200-225, 121-250 etc. The term “about” has its ordinary meaning of approximately and may be determined in context by experimental variability. In case of doubt, “about” means a variation within 5% of a stated numerical value.

The term “polynucleotide” corresponds to either double-stranded or single-stranded cDNA or genomic DNA or RNA, containing at least 10 contiguous nucleotides. Single stranded polynucleic acid sequences are always represented in the current invention from the 5′ end to the 3′ end. Polynucleic acids according to the invention may be prepared by any method known in the art for preparing polynucleic acids (e.g. the phosphodiester method for synthesizing oligonucleotides as described by Agarwal et al. (1972), the phosphotriester method of Hsiung et al. (1979), or the automated diethylphosphoroamidite method of Baeucage et al. (1981)). Alternatively, the polynucleic acids of the invention may be isolated fragments of naturally occurring or cloned DNA or RNA.

The term “oligonucleotide” refers to a single stranded nucleic acid comprising two or more nucleotides, and less than 300 nucleotides. The exact size of an oligonucleotide depends on the ultimate function or use of said oligonucleotide. For use as a probe or primer the oligonucleotides are preferably about 5-50 nucleotides long.

The oligonucleotides and polynucleotides according to the present invention can be formed by cloning of recombinant plasmids containing inserts including the corresponding nucleotide sequences, if need be by cleaving the latter out from the cloned plasmids upon using the adequate nucleases and recovering them, e.g. by fractionation according to molecular weight. The probes according to the present invention can also be synthesized chemically, e.g. by automatic synthesis on commercial instruments sold by a variety of manufacturers.

The nucleotides as used in the present invention may, in certain aspects, be ribonucleotides, deoxyribonucleotides and modified nucleotides such as inosine or nucleotides containing modified groups which do not essentially alter their hybridisation characteristics. Moreover, it is obvious to the man skilled in the art that any of the below-specified probes can be used as such, or in their complementary form, or in their RNA form (wherein T is replaced by U).

The oligonucleotides used as primers or probes may also comprise or consist of nucleotide analogues such as phosphorothioates (Matsukura et al., 1987). alkylphosphorothioiates (Miller et al., 1979) or peptide nucleic acids (Nielsen et al., 1991; Nielsen et al., 1993) or may contain intercalating agents (Asseline et al., 1984).

The term “probe” refers to single stranded sequencespecific oligonucleotides which have a sequence which is sufficiently complementary to hybridize to the target sequence to be detected. Preferably said probes are 70%, 80%, 90%, or more than 95% homologous to the exact complement of the target sequence to be detected. These target sequences are either genomic DNA or messenger RNA, or amplified versions thereof. Preferably, these probes are about 5 to 50 nucleotides long, more preferably from about 10 to 30 nucleotides.

The term “hybridizes to” refers to preferably stringent hybridizations conditions, allowing hybridisation between complementary nucleic acid sequences showing at least 90%, 95% or more homology with each other.

The term “primer” refers to a single stranded DNA oligonucleotide sequence capable of acting as a point of initiation for synthesis of a primer extension product which 5 is complementary to the nucleic acid strand to be copied. The length and the sequence of the primer must be such that they allow to prime the synthesis of the extension products. Preferably the primer is about 5-50 nucleotides long. Specific length and sequence will depend on the complexity of the required DNA or RNA targets, as well as on the conditions of primer use such as temperature and ionic strength. The fact that amplification primers do not have to match exactly with the corresponding template sequence to warrant proper amplification is amply documented in the literature. The amplification method used can be either polymerase chain reaction, target polynucleotide amplification methods such as self-sustained sequence replication (3SR) and strand-displacement amplification (SDA); methods based on amplification of a signal attached to the target polynucleotide, such as “branched chain” DNA amplification; methods based on amplification of probe DNA, such as ligase chain reaction (LCR) and QB replicase amplification (QBR); transcription-based methods, such as ligation activated transcription (LAT), nucleic acid sequence-based amplification (NASBA), amplification under the trade name INVADER, and transcription-mediated amplification (TMA); and various other amplification methods, such as repair chain reaction (RCR) and cycling probe reaction (CPR). Preferred methods can be multiplexed, i.e. a number of amplifications of different sequences can be run in the same reaction mixture at the same time.

The term “complementary” nucleic acids as used in the current invention means that the nucleic acid sequences can form a perfect base paired double helix with each other.

The term “FFPE” refers to formalin-fixed, paraffin-embedded (FFPE) tissue samples. Commercial solutions of formadehyde in water are commonly called formalin. Formalin preserves or fixes tissue or cells by reversibly cross-linking primary amino groups in proteins with other nearby nitrogen atoms in protein or DNA through a —CH₂— linkage.

Tissue samples are typically placed into molds along with liquid embedding material (such as agar, gelatine, or wax) which is then hardened. This is achieved by cooling in the case of paraffin wax and heating (curing) in the case of the epoxy resins. The acrylic resins are polymerised by heat, ultraviolet light, or chemical catalysts. The hardened blocks containing the tissue samples are then ready to be sectioned.

Another aldehyde that can be used for fixation is glutaraldehyde. It operates in a similar way to formaldehyde by causing deformation of the alpha-helix structures in proteins. However, glutaraldehyde is a larger molecule, and so its rate of diffusion across membranes is slower than formaldehyde.

Samples that may be used in the present invention include medical samples, forensic samples, museum or archeological samples, and other archival collections, which need not be FFPE preserved. There are many preservation methods that have been applied to tissues, including alcohol preservation, formalin treatment, freezing and sequestration in waxes and other materials. In addition, forensic or archeological samples may contain degraded ssDNA that has not been consciously preserved at all.

The term “5′ exonuclease” or “5′ end nuclease” refers to an enzyme that has activity 5′ to 3′ direction to remove a single stranded DNA having a 5′ end. It may do this through exonuclease or endonuclease activity, i.e. cleavage at a point where the ssDNA separates from its complementary strand. The 5′ exonuclease enzymes used herein preferably degrade single stranded DNA, not double stranded DNA. The preferred 5′ exonuclease is a DNA polymerase that has the ability to cleave a DNA hairpin where a 5′ end of DNA to be cleaved is a single strand adjacent to a double strand, which may result from formation of an exogenous duplex, such as hybridization to a primer. For details, see Lyamichev et al. “Structure-Specific Endonucleolytic Cleavage of Nucleic Acids by Eubacterial DNA Polymerases,” Science 260:778-783 (1993), describing this activity in DNAP-Ecl and DNAP-Taq (from Thermus aquaticus) polymerases.

The term “3′ exonuclease” or “3′ end nuclease” refers to an enzyme having activity in the 3′ to 5′ direction to remove a single stranded DNA portion having a 3′ end. As with the 5′ exonuclease, the enzyme will only act on ssDNA and may do this by either exonuclease or endonuclease activity. This activity is found as DNA proofreading in certain DNA polymerases. It allows the enzyme to check each nucleotide during DNA synthesis, and excise mismatched nucleotides in the 3′ to 5′ direction. The proofreading domain also enables a polymerase to remove unpaired 3′ overhanging nucleotides to create blunt ends. Protocols such as high-fidelity PCR, 3′ overhang polishing and high-fidelity second strand synthesis require the presence of a 3′→5′ exonuclease.

The preferred 3′ exonuclease is Exo I. Exonuclease I (Exo I), the product of the sbcB gene of E. coli, is an exodeoxyribonuclease that hydrolyzes single-stranded (ss)DNA stepwise in a 3′ to 5′ direction. 1-3 Hydrolysis generates deoxyribonucleoside 5′-monophosphates and a terminal dinucleotide diphosphate. The enzyme requires magnesium (optimal Mg++ concentration is 10 mM) and the presence of a free 3′-hydroxyl terminus. Exonuclease I is active under a wide variety of buffer conditions, allowing addition of the enzyme directly into most reaction mixes. Heat inactivation results from incubation at 80° C. for 15 minutes.

The term “ligase” refers to an enzyme that catalyzes formation of a phosphodiester bond between the 5′ phosphate of one strand of DNA and the 3′ hydroxyl of the other. This enzyme is used to covalently link or ligate fragments of DNA together. An example of a DNA ligase is one derived from the T4 bacteriophage. T4 DNA ligase requires ATP as a cofactor. The presently preferred ligase is Ampligase® ligase (registered trademark of Epicentre Technologies), a thermostable DNA ligase that catalyzes NAD-dependent ligation of adjacent 3′-hydroxylated and 5′-phosphorylated termini in duplex DNA structures that are stable at high temperatures.

For convenience, certain polynucleotides are referred to herein as “capture probes,” meaning single stranded polynucleotides of relatively small size, e.g. 40-4000 bases, which are prepared (e.g. synthetically) to contain defined features. These include certain “universal” sequences, which are so designated because they are essentially identical as between different polynucleotides designed for the stated purpose, whereas other sequences in the capture probes will vary among a number of different possibilities to capture different targets. That is, the capture probes contain a “universal probe sequence” which contains a single sequence common to all capture probes. In this way, the “universal polynucleotides” may have a single sequence that is complementary to the universal sequence in the capture probes.

### EXAMPLES

### Example 1

### Oligonucleotide Design, Target DNA Capture, and Sequencing

### Samples

Genomic DNA from NA18507 was obtained from Corriel Cell Repositories. Intestinal tissue samples were obtained from under an IRB protocol approved by Stanford University. These samples were either immediately snap frozen in liquid nitrogen and stored at −80° C. or preserved as formalin-fixed, paraffin-embedded (FFPE) blocks. Total nucleic acids were extracted from the flash-frozen tissue using the SQ DNA/RNA/Protein Kit from Omega Bio-Tek. Following complete RNase A digestion, the DNA (herein referred as dsDNA) was analyzed by argarose gel electrophoresis and quantified by a fluorescence assay using SYBR Gold (Invitrogen). For FFPE samples, DNA was isolated using the BiOstic® FFPE Tissue DNA Isolation Kit from Mo Bio Laboratories. The quantity and quality of the preparations were by OD260 and qPCR analysis across 3 different genomic loci. Only single stranded DNA (ssDNA) samples with a difference in Ct values of equal or less than 4.0 or approximately 15% genome equivalence between the flash-frozen and FFPE samples were used for subsequent analysis.

### Capture Polynucleotides and Sequence Listing

Capture polynucleotides with the properties optimal for FFPE capture were chosen from a larger, previously described set (Natsoulis et al. 2011, Ref. 9). As disclosed there, the oligonucleotide sequences can be downloaded from the Human OligoExome, a database which provides gene exons annotated by the Consensus Coding Sequencing Project (CCDS). The database is available at oligoexome.Stanford.edu. 628 capture oligonucleotides resulting in amplicons ranging from 150 to 250 bp were chosen from this set. 2,512 sequences containing sequences of the 5′ targeting arm, 3′ targeting arm, amplicon, and target oligonucleotide for each of the 628 capture oligonucleotides were compiled. Targeting arms were positioned in regions without SNPs per dbSNP. Details on the design parameters and on the capture characteristics of the targeting arms are provided by Natsoulis et al. [9].

The accompanying sequence listing sets forth the sequences of the 5′ targeting arm, the 3′ targeting arm, the amplicon sequence and the universal oligonucleotide used, including uridine substitutions for the 628 capture probes used in the examples. In the table below, Column 1 is the chromosome number targeted; column 2 is the position of the 5′ end of the targeted sequence; col. 3 is the polarity of the targeted strand; column 4 (SEQ ID NOs) is sequence of the 20 bp 5′ targeting arm; column 5 (SEQ ID NOs) is the sequence of the 3′ 20 bp selector; column 6 (SEQ ID NOs) lists the sequences of the amplicons and column 7 (SEQ ID NOs) lists the sequences of the targeting oligonucleotides (“universal probes”) including uridine substitutions; and column 8 is the identifier (which may also be checked at the Stanford OligoExome web site).

| | | | Col. 4 | Col. 5 | Col. 6 | Col. 7 | |
|---|---|---|---|---|---|---|---|
| Col. 1 | Col. 2 | Col. 3 | SEQ ID NO: | SEQ ID NO: | SEQ ID NO: | SEQ ID NO: | Col. 8 |
| 2 | 197978441 | minus | 1 | 2 | 3 | 4 | SF3B1_ROI_10 |
| 7 | 81531606 | minus | 5 | 6 | 7 | 8 | CACNA2D1_ROI_9 |
| 3 | 73516265 | minus | 9 | 10 | 11 | 12 | PDZRN3_ROI_10 |
| 18 | 32189513 | minus | 13 | 14 | 15 | 16 | FHOD3_ROI_2 |
| 7 | 81481731 | minus | 17 | 18 | 19 | 20 | CACNA2D1_ROI_13 |
| 4 | 1777300 | minus | 21 | 22 | 23 | 24 | FGFR3_ROI_8 |
| 6 | 3022048 | plus | 25 | 26 | 27 | 28 | RIPK1_ROI_1 |
| 1 | 56934140 | minus | 29 | 30 | 31 | 32 | PRKAA2_ROI_6 |
| 22 | 28364929 | plus | 33 | 34 | 35 | 36 | NF2_ROI_3 |
| 15 | 20512084 | plus | 37 | 38 | 39 | 40 | CYFIP1_ROI_15 |
| 7 | 98397711 | plus | 41 | 42 | 43 | 44 | TRRAP_ROI_41 |
| 7 | 98401105 | plus | 45 | 46 | 47 | 48 | TRRAP_ROI_43 |
| 6 | 3049527 | minus | 49 | 50 | 51 | 52 | RIPK1_ROI_7 |
| 18 | 57318446 | plus | 53 | 54 | 55 | 56 | CDH20_ROI_3 |
| 20 | 61808885 | minus | 57 | 58 | 59 | 60 | ARFRP1_ROI_1 |
| 19 | 10959598 | minus | 61 | 62 | 63 | 64 | SMARCA4_ROI_5 |
| 2 | 106813126 | minus | 65 | 66 | 67 | 68 | ST6GAL2_ROI_4 |
| 15 | 65266899 | minus | 69 | 70 | 71 | 72 | SMAD3_ROI_8 |
| 18 | 51168635 | minus | 73 | 74 | 75 | 76 | TCF4_ROI_7 |
| 12 | 50666736 | minus | 77 | 78 | 79 | 80 | ACVR1B_ROI_7 |
| 3 | 89472909 | minus | 81 | 82 | 83 | 84 | EPHA3_ROI_4 |
| 23 | 69635044 | minus | 85 | 86 | 87 | 88 | DLG3_ROI_17 |
| 7 | 148157809 | minus | 89 | 90 | 91 | 92 | EZH2_ROI_4 |
| 7 | 98391555 | plus | 93 | 94 | 95 | 96 | TRRAP_ROI_37 |
| 8 | 113461681 | minus | 97 | 98 | 99 | 100 | CSMD3_ROI_39 |
| 10 | 55296258 | plus | 101 | 102 | 103 | 104 | PCDH15_ROI_26 |
| 4 | 1778491 | plus | 105 | 106 | 107 | 108 | FGFR3_ROI_12 |
| 1 | 6117503 | minus | 109 | 110 | 111 | 112 | CHD5_ROI_16 |
| 22 | 28400927 | minus | 113 | 114 | 115 | 116 | NF2_ROI_13 |
| 11 | 85666939 | plus | 117 | 118 | 119 | 120 | EED_ROI_12 |
| 19 | 10982204 | minus | 121 | 122 | 123 | 124 | SMARCA4_ROI_14 |
| 5 | 112129928 | minus | 125 | 126 | 127 | 128 | APC_ROI_2 |
| 15 | 65269671 | minus | 129 | 130 | 131 | 132 | SMAD3_ROI_9 |
| 1 | 11115574 | plus | 133 | 134 | 135 | 136 | FRAP1_ROI_35 |
| 19 | 35000025 | minus | 137 | 138 | 139 | 140 | CCNE1_ROI_4 |
| 20 | 35461947 | minus | 141 | 142 | 143 | 144 | SRC_ROI_7 |
| 11 | 107633539 | minus | 145 | 146 | 147 | 148 | ATM_ROI_13 |
| 1 | 173603051 | minus | 149 | 150 | 151 | 152 | TNR_ROI_8 |
| 1 | 6093131 | minus | 153 | 154 | 155 | 156 | CHD5_ROI_34 |
| 7 | 140124110 | plus | 157 | 158 | 159 | 160 | BRAF_ROI_12 |
| 18 | 46838465 | minus | 161 | 162 | 163 | 164 | SMAD4_ROI_5 |
| 23 | 85954365 | plus | 165 | 166 | 167 | 168 | DACH2_ROI_8 |
| 3 | 132282116 | minus | 169 | 170 | 171 | 172 | NEK11_ROI_2 |
| 23 | 69638588 | plus | 173 | 174 | 175 | 176 | DLG3_ROI_22 |
| 15 | 20487399 | minus | 177 | 178 | 179 | 180 | CYFIP1_ROI_6 |
| 15 | 20479945 | minus | 181 | 182 | 183 | 184 | CYFIP1_ROI_3 |
| 6 | 80806019 | minus | 185 | 186 | 187 | 188 | TTK_ROI_17 |
| 2 | 197966150 | minus | 189 | 190 | 191 | 192 | SF3B1_ROI_21 |
| 12 | 77093105 | minus | 193 | 194 | 195 | 196 | NAV3_ROI_25 |
| 4 | 55260662 | minus | 197 | 198 | 199 | 200 | KIT_ROI_4 |
| 1 | 11110373 | minus | 201 | 202 | 203 | 204 | FRAP1_ROI_41 |
| 23 | 122364376 | plus | 205 | 206 | 207 | 208 | GRIA3_ROI_8 |
| 8 | 113771268 | minus | 209 | 210 | 211 | 212 | CSMD3_ROI_15 |
| 3 | 89604424 | minus | 213 | 214 | 215 | 216 | EPHA3_ROI_16 |
| 2 | 179352082 | minus | 217 | 218 | 219 | 220 | TTN_ROI_22 |
| 5 | 24524011 | minus | 221 | 222 | 223 | 224 | CDH10_ROI_11 |
| 11 | 64331720 | minus | 225 | 226 | 227 | 228 | MEN1_ROI_3 |
| 19 | 11013228 | minus | 229 | 230 | 231 | 232 | SMARCA4_ROI_29 |
| 23 | 69585530 | minus | 233 | 234 | 235 | 236 | DLG3_ROI_2 |
| 11 | 107619747 | minus | 237 | 238 | 239 | 240 | ATM_ROI_4 |
| 1 | 74782245 | minus | 241 | 242 | 243 | 244 | TNNI3K_ROI_23 |
| 10 | 42922016 | minus | 245 | 246 | 247 | 248 | RET_ROI_5 |
| 2 | 79990425 | minus | 249 | 250 | 251 | 252 | CTNNA2_ROI_6 |
| 2 | 197978280 | plus | 253 | 254 | 255 | 256 | SF3B1_ROI_10 |
| 10 | 89714874 | plus | 257 | 258 | 259 | 260 | PTEN_ROI_9 |
| 7 | 55234021 | minus | 261 | 262 | 263 | 264 | EGFR_ROI_24 |
| 16 | 23629188 | plus | 265 | 266 | 267 | 268 | ERN2_ROI_3 |
| 15 | 20542653 | minus | 269 | 270 | 271 | 272 | CYFIP1_ROI_22 |
| 18 | 41921446 | minus | 273 | 274 | 275 | 276 | ATP5A1_ROI_6 |
| 23 | 69590881 | minus | 277 | 278 | 279 | 280 | DLG3_ROI_10 |
| 5 | 24628976 | plus | 281 | 282 | 283 | 284 | CDH10_ROI_1 |
| 18 | 49086077 | minus | 285 | 286 | 287 | 288 | DCC_ROI_13 |
| 19 | 10999310 | plus | 289 | 290 | 291 | 292 | SMARCA4_ROI_23 |
| 17 | 10377190 | minus | 293 | 294 | 295 | 296 | MYH2_ROI_13 |
| 2 | 179372871 | minus | 297 | 298 | 299 | 300 | TTN_ROI_4 |
| 13 | 31804798 | plus | 301 | 302 | 303 | 304 | BRCA2_ROI_8 |
| 7 | 151467266 | minus | 305 | 306 | 307 | 308 | MLL3_ROI_56 |
| 1 | 173559224 | minus | 309 | 310 | 311 | 312 | TNR_ROI_21 |
| 18 | 32189350 | plus | 313 | 314 | 315 | 316 | FHOD3_ROI_2 |
| 10 | 55332859 | plus | 317 | 318 | 319 | 320 | PCDH15_ROI_25 |
| 11 | 107723290 | minus | 321 | 322 | 323 | 324 | ATM_ROI_56 |
| 8 | 113392559 | minus | 325 | 326 | 327 | 328 | CSMD3_ROI_51 |
| 8 | 37809872 | minus | 329 | 330 | 331 | 332 | GPR124_ROI_9 |
| 19 | 10993251 | plus | 333 | 334 | 335 | 336 | SMARCA4_ROI_18 |
| 23 | 47309470 | minus | 337 | 338 | 339 | 340 | ARAF_ROI_3 |
| 13 | 31810986 | plus | 341 | 342 | 343 | 344 | BRCA2_ROI_9 |
| 12 | 77039778 | minus | 345 | 346 | 347 | 348 | NAV3_ROI_16 |
| 12 | 130056498 | minus | 349 | 350 | 351 | 352 | GPR133_ROI_11 |
| 2 | 197980963 | minus | 353 | 354 | 355 | 356 | SF3B1_ROI_9 |
| 6 | 3056175 | minus | 357 | 358 | 359 | 360 | RIPK1_ROI_9 |
| 12 | 119918586 | minus | 361 | 362 | 363 | 364 | HNF1A_ROI_5 |
| 7 | 81479256 | plus | 365 | 366 | 367 | 368 | CACNA2D1_ROI_15 |
| 23 | 85856366 | minus | 369 | 370 | 371 | 372 | DACH2_ROI_6 |
| 20 | 35461782 | plus | 373 | 374 | 375 | 376 | SRC_ROI_7 |
| 17 | 7518320 | minus | 377 | 378 | 379 | 380 | TP53_ROI_5 |
| 20 | 35464392 | minus | 381 | 382 | 383 | 384 | SRC_ROI_9 |
| 7 | 148157603 | plus | 385 | 386 | 387 | 388 | EZH2_ROI_4 |
| 7 | 113346109 | minus | 389 | 390 | 391 | 392 | PPP1R3A_ROI_1 |
| 4 | 55286630 | plus | 393 | 394 | 395 | 396 | KIT_ROI_9 |
| 10 | 55257300 | minus | 397 | 398 | 399 | 400 | PCDH15_ROI_31 |
| 1 | 6092590 | minus | 401 | 402 | 403 | 404 | CHD5_ROI_35 |
| 2 | 1405894 | minus | 405 | 406 | 407 | 408 | TPO_ROI_2 |
| 13 | 31842389 | plus | 409 | 410 | 411 | 412 | BRCA2_ROI_17 |
| 1 | 173639039 | minus | 413 | 414 | 415 | 416 | TNR_ROI_2 |
| 18 | 20896668 | minus | 417 | 418 | 419 | 420 | ZNF521_ROI_7 |
| 7 | 81533723 | minus | 421 | 422 | 423 | 424 | CACNA2D1_ROI_8 |
| 4 | 1777450 | minus | 425 | 426 | 427 | 428 | FGFR3_ROI_8 |
| 1 | 173598272 | plus | 429 | 430 | 431 | 432 | TNR_ROI_12 |
| 5 | 112182432 | plus | 433 | 434 | 435 | 436 | APC_ROI_9 |
| 20 | 29589493 | plus | 437 | 438 | 439 | 440 | HM13_ROI_3 |
| 1 | 74569805 | minus | 441 | 442 | 443 | 444 | TNNI3K_ROI_6 |
| 4 | 138672620 | minus | 445 | 446 | 447 | 448 | PCDH18_ROI_1 |
| 3 | 180405054 | minus | 449 | 450 | 451 | 452 | PIK3CA_ROI_5 |
| 6 | 80778400 | minus | 453 | 454 | 455 | 456 | TTK_ROI_6 |
| 12 | 130037831 | minus | 457 | 458 | 459 | 460 | GPR133_ROI_5 |
| 2 | 179332128 | minus | 461 | 462 | 463 | 464 | TTN_ROI_37 |
| 6 | 3028510 | minus | 465 | 466 | 467 | 468 | RIPK1_ROI_4 |
| 8 | 113881663 | minus | 469 | 470 | 471 | 472 | CSMD3_ROI_14 |
| 7 | 98327833 | plus | 473 | 474 | 475 | 476 | TRRAP_ROI_4 |
| 20 | 29566181 | minus | 477 | 478 | 479 | 480 | HM13_ROI_1 |
| 8 | 113368401 | minus | 481 | 482 | 483 | 484 | CSMD3_ROI_59 |
| 19 | 1002034 | minus | 485 | 486 | 487 | 488 | ABCA7_ROI_14 |
| 23 | 122366090 | plus | 489 | 490 | 491 | 492 | GRIA3_ROI_10 |
| 13 | 31798088 | plus | 493 | 494 | 495 | 496 | BRCA2_ROI_4 |
| 19 | 10967739 | plus | 497 | 498 | 499 | 500 | SMARCA4_ROI_9 |
| 15 | 20487151 | plus | 501 | 502 | 503 | 504 | CYFIP1_ROI_6 |
| 23 | 122426247 | plus | 505 | 506 | 507 | 508 | GRIA3_ROI_13 |
| 15 | 20512312 | minus | 509 | 510 | 511 | 512 | CYFIP1_ROI_15 |
| 11 | 107633350 | minus | 513 | 514 | 515 | 516 | ATM_ROI_13 |
| 3 | 49874078 | plus | 517 | 518 | 519 | 520 | CAMKV_ROI_3 |
| 17 | 35134965 | minus | 521 | 522 | 523 | 524 | ERBB2_ROI_18 |
| 1 | 173615171 | minus | 525 | 526 | 527 | 528 | TNR_ROI_7 |
| 23 | 122215055 | minus | 529 | 530 | 531 | 532 | GRIA3_ROI_3 |
| 19 | 998634 | minus | 533 | 534 | 535 | 536 | ABCA7_ROI_11 |
| 19 | 10990483 | plus | 537 | 538 | 539 | 540 | SMARCA4_ROI_16 |
| 18 | 49102265 | plus | 541 | 542 | 543 | 544 | DCC_ROI_14 |
| 5 | 14540423 | plus | 545 | 546 | 547 | 548 | TRIO_ROI_47 |
| 6 | 3022200 | minus | 549 | 550 | 551 | 552 | RIPK1_ROI_1 |
| 4 | 55260490 | plus | 553 | 554 | 555 | 556 | KIT_ROI_4 |
| 7 | 98371213 | minus | 557 | 558 | 559 | 560 | TRRAP_ROI_26 |
| 6 | 70138988 | plus | 561 | 562 | 563 | 564 | BAI3_ROI_28 |
| 2 | 47863727 | plus | 565 | 566 | 567 | 568 | MSH6_ROI_1 |
| 15 | 20542423 | plus | 569 | 570 | 571 | 572 | CYFIP1_ROI_22 |
| 20 | 35465175 | minus | 573 | 574 | 575 | 576 | SRC_ROI_11 |
| 19 | 11030314 | plus | 577 | 578 | 579 | 580 | SMARCA4_ROI_31 |
| 12 | 76939508 | plus | 581 | 582 | 583 | 584 | NAV3_ROI_9 |
| 2 | 179374960 | plus | 585 | 586 | 587 | 588 | TTN_ROI_2 |
| 17 | 10375909 | minus | 589 | 590 | 591 | 592 | MYH2_ROI_14 |
| 5 | 14452158 | minus | 593 | 594 | 595 | 596 | TRIO_ROI_29 |
| 18 | 32410317 | minus | 597 | 598 | 599 | 600 | FHOD3_ROI_6 |
| 3 | 132430096 | minus | 601 | 602 | 603 | 604 | NEK11_ROI_12 |
| 8 | 113654983 | minus | 605 | 606 | 607 | 608 | CSMD3_ROI_25 |
| 7 | 98440747 | minus | 609 | 610 | 611 | 612 | TRRAP_ROI_63 |
| 6 | 3030397 | minus | 613 | 614 | 615 | 616 | RIPK1_ROI_5 |
| 19 | 1008764 | plus | 617 | 618 | 619 | 620 | ABCA7_ROI_27 |
| 17 | 26687327 | plus | 621 | 622 | 623 | 624 | NF1_ROI_41 |
| 23 | 69582111 | minus | 625 | 626 | 627 | 628 | DLG3_ROI_1 |
| 7 | 151480573 | plus | 629 | 630 | 631 | 632 | MLL3_ROI_48 |
| 1 | 58744170 | plus | 633 | 634 | 635 | 636 | OMA1_ROI_6 |
| 8 | 113306238 | plus | 637 | 638 | 639 | 640 | CSMD3_ROI_72 |
| 17 | 26688035 | minus | 641 | 642 | 643 | 644 | NF1_ROI_42 |
| 5 | 24545302 | plus | 645 | 646 | 647 | 648 | CDH10_ROI_6 |
| 19 | 10325887 | minus | 649 | 650 | 651 | 652 | TYK2_ROI_13 |
| 6 | 41663548 | minus | 653 | 654 | 655 | 656 | FOXP4_ROI_7 |
| 1 | 6092956 | plus | 657 | 658 | 659 | 660 | CHD5_ROI_34 |
| 23 | 70600532 | minus | 661 | 662 | 663 | 664 | TAF1_ROI_36 |
| 1 | 6089133 | minus | 665 | 666 | 667 | 668 | CHD5_ROI_37 |
| 18 | 51087922 | minus | 669 | 670 | 671 | 672 | TCF4_ROI_10 |
| 1 | 173598457 | plus | 673 | 674 | 675 | 676 | TNR_ROI_12 |
| 15 | 20549735 | plus | 677 | 678 | 679 | 680 | CYFIP1_ROI_25 |
| 19 | 1004178 | plus | 681 | 682 | 683 | 684 | ABCA7_ROI_18 |
| 6 | 41663374 | minus | 685 | 686 | 687 | 688 | FOXP4_ROI_7 |
| 22 | 28384229 | minus | 689 | 690 | 691 | 692 | NF2_ROI_7 |
| 8 | 113335653 | minus | 693 | 694 | 695 | 696 | CSMD3_ROI_64 |
| 1 | 6110543 | plus | 697 | 698 | 699 | 700 | CHD5_ROI_22 |
| 8 | 114457993 | plus | 701 | 702 | 703 | 704 | CSMD3_ROI_2 |
| 17 | 26532904 | minus | 705 | 706 | 707 | 708 | NF1_ROI_7 |
| 11 | 107626580 | plus | 709 | 710 | 711 | 712 | ATM_ROI_8 |
| 8 | 113598298 | minus | 713 | 714 | 715 | 716 | CSMD3_ROI_29 |
| 3 | 73535839 | plus | 717 | 718 | 719 | 720 | PDZRN3_ROI_4 |
| 12 | 130056320 | plus | 721 | 722 | 723 | 724 | GPR133_ROI_11 |
| 14 | 102504422 | minus | 725 | 726 | 727 | 728 | CDC42BPB_ROI_15 |
| 10 | 55370482 | plus | 729 | 730 | 731 | 732 | PCDH15_ROI_23 |
| 11 | 85638836 | plus | 733 | 734 | 735 | 736 | EED_ROI_2 |
| 16 | 67406769 | plus | 737 | 738 | 739 | 740 | CDH1_ROI_10 |
| 5 | 14431209 | minus | 741 | 742 | 743 | 744 | TRIO_ROI_20 |
| 2 | 179365142 | minus | 745 | 746 | 747 | 748 | TTN_ROI_8 |
| 2 | 179377374 | plus | 749 | 750 | 751 | 752 | TTN_ROI_1 |
| 12 | 130186634 | minus | 753 | 754 | 755 | 756 | GPR133_ROI_21 |
| 2 | 179363060 | minus | 757 | 758 | 759 | 760 | TTN_ROI_10 |
| 4 | 1771021 | minus | 761 | 762 | 763 | 764 | FGFR3_ROI_2 |
| 2 | 80669895 | minus | 765 | 766 | 767 | 768 | CTNNA2_ROI_14 |
| 7 | 113307388 | minus | 769 | 770 | 771 | 772 | PPP1R3A_ROI_3 |
| 23 | 70246581 | plus | 773 | 774 | 775 | 776 | IL2RG_ROI_4 |
| 19 | 10325294 | minus | 777 | 778 | 779 | 780 | TYK2_ROI_14 |
| 12 | 119911190 | minus | 781 | 782 | 783 | 784 | HNF1A_ROI_2 |
| 18 | 48121006 | plus | 785 | 786 | 787 | 788 | DCC_ROI_1 |
| 5 | 112144471 | minus | 789 | 790 | 791 | 792 | APC_ROI_5 |
| 1 | 6106725 | minus | 793 | 794 | 795 | 796 | CHD5_ROI_28 |
| 4 | 107373641 | minus | 797 | 798 | 799 | 800 | MGC16169_ROI_14 |
| 14 | 102480548 | minus | 801 | 802 | 803 | 804 | CDC42BPB_ROI_29 |
| 2 | 179347763 | plus | 805 | 806 | 807 | 808 | TTN_ROI_26 |
| 6 | 69741756 | minus | 809 | 810 | 811 | 812 | BAI3_ROI_8 |
| 1 | 58777086 | minus | 813 | 814 | 815 | 816 | OMA1_ROI_1 |
| 1 | 6125223 | minus | 817 | 818 | 819 | 820 | CHD5_ROI_13 |
| 8 | 114458170 | minus | 821 | 822 | 823 | 824 | CSMD3_ROI_2 |
| 12 | 130004814 | plus | 825 | 826 | 827 | 828 | GPR133_ROI_1 |
| 8 | 113771425 | minus | 829 | 830 | 831 | 832 | CSMD3_ROI_15 |
| 19 | 10991347 | minus | 833 | 834 | 835 | 836 | SMARCA4_ROI_17 |
| 19 | 10984475 | plus | 837 | 838 | 839 | 840 | SMARCA4_ROI_15 |
| 2 | 106789533 | minus | 841 | 842 | 843 | 844 | ST6GAL2_ROI_5 |
| 19 | 10956019 | minus | 845 | 846 | 847 | 848 | SMARCA4_ROI_1 |
| 11 | 85665798 | minus | 849 | 850 | 851 | 852 | EED_ROI_10 |
| 6 | 3055886 | plus | 853 | 854 | 855 | 856 | RIPK1_ROI_9 |
| 12 | 25253986 | minus | 857 | 858 | 859 | 860 | KRAS_ROI_5 |
| 9 | 93528815 | minus | 861 | 862 | 863 | 864 | ROR2_ROI_8 |
| 1 | 64415549 | plus | 865 | 866 | 867 | 868 | ROR1_ROI_9 |
| 7 | 98412584 | minus | 869 | 870 | 871 | 872 | TRRAP_ROI_50 |
| 6 | 3028223 | minus | 873 | 874 | 875 | 876 | RIPK1_ROI_4 |
| 17 | 35134678 | plus | 877 | 878 | 879 | 880 | ERBB2_ROI_18 |
| 3 | 132429912 | plus | 881 | 882 | 883 | 884 | NEK11_ROI_12 |
| 15 | 20506689 | minus | 885 | 886 | 887 | 888 | CYFIP1_ROI_12 |
| 11 | 107708549 | plus | 889 | 890 | 891 | 892 | ATM_ROI_50 |
| 6 | 41664415 | minus | 893 | 894 | 895 | 896 | FOXP4_ROI_8 |
| 12 | 119921304 | plus | 897 | 898 | 899 | 900 | HNF1A_ROI_8 |
| 18 | 32578121 | minus | 901 | 902 | 903 | 904 | FHOD3_ROI_20 |
| 11 | 107701845 | plus | 905 | 906 | 907 | 908 | ATM_ROI_44 |
| 5 | 14422333 | plus | 909 | 910 | 911 | 912 | TRIO_ROI_18 |
| 7 | 98345465 | plus | 913 | 914 | 915 | 916 | TRRAP_ROI_14 |
| 15 | 20498252 | plus | 917 | 918 | 919 | 920 | CYFIP1_ROI_10 |
| 15 | 20479614 | minus | 921 | 922 | 923 | 924 | CYFIP1_ROI_2 |
| 15 | 20492019 | plus | 925 | 926 | 927 | 928 | CYFIP1_ROI_8 |
| 15 | 20554394 | minus | 929 | 930 | 931 | 932 | CYFIP1_ROI_28 |
| 12 | 25269940 | minus | 933 | 934 | 935 | 936 | KRAS_ROI_3 |
| 3 | 49874248 | minus | 937 | 938 | 939 | 940 | CAMKV_ROI_3 |
| 1 | 64397295 | minus | 941 | 942 | 943 | 944 | ROR1_ROI_8 |
| 17 | 10379373 | minus | 945 | 946 | 947 | 948 | MYH2_ROI_12 |
| 7 | 151486731 | plus | 949 | 950 | 951 | 952 | MLL3_ROI_43 |
| 16 | 23628844 | plus | 953 | 954 | 955 | 956 | ERN2_ROI_4 |
| 17 | 26604010 | minus | 957 | 958 | 959 | 960 | NF1_ROI_31 |
| 23 | 70518168 | plus | 961 | 962 | 963 | 964 | TAF1_ROI_9 |
| 7 | 151533118 | minus | 965 | 966 | 967 | 968 | MLL3_ROI_25 |
| 8 | 114360074 | minus | 969 | 970 | 971 | 972 | CSMD3_ROI_4 |
| 3 | 41252069 | plus | 973 | 974 | 975 | 976 | CTNNB1_ROI_8 |
| 10 | 42924641 | minus | 977 | 978 | 979 | 980 | RET_ROI_6 |
| 1 | 74473574 | plus | 981 | 982 | 983 | 984 | TNNI3K_ROI_1 |
| 17 | 26681290 | plus | 985 | 986 | 987 | 988 | NF1_ROI_39 |
| 5 | 14560333 | minus | 989 | 990 | 991 | 992 | TRIO_ROI_55 |
| 8 | 113385976 | plus | 993 | 994 | 995 | 996 | CSMD3_ROI_53 |
| 8 | 113940635 | minus | 997 | 998 | 999 | 1000 | CSMD3_ROI_12 |
| 10 | 42943416 | plus | 1001 | 1002 | 1003 | 1004 | RET_ROI_20 |
| 20 | 35463459 | minus | 1005 | 1006 | 1007 | 1008 | SRC_ROI_8 |
| 1 | 64288796 | plus | 1009 | 1010 | 1011 | 1012 | ROR1_ROI_4 |
| 11 | 107646851 | plus | 1013 | 1014 | 1015 | 1016 | ATM_ROI_17 |
| 6 | 41664210 | plus | 1017 | 1018 | 1019 | 1020 | FOXP4_ROI_8 |
| 12 | 77108036 | plus | 1021 | 1022 | 1023 | 1024 | NAV3_ROI_33 |
| 16 | 23629030 | minus | 1025 | 1026 | 1027 | 1028 | ERN2_ROI_4 |
| 11 | 107695741 | plus | 1029 | 1030 | 1031 | 1032 | ATM_ROI_41 |
| 7 | 98316772 | minus | 1033 | 1034 | 1035 | 1036 | TRRAP_ROI_1 |
| 22 | 28381453 | plus | 1037 | 1038 | 1039 | 1040 | NF2_ROI_6 |
| 1 | 6114357 | minus | 1041 | 1042 | 1043 | 1044 | CHD5_ROI_18 |
| 18 | 49177776 | minus | 1045 | 1046 | 1047 | 1048 | DCC_ROI_18 |
| 20 | 35463269 | plus | 1049 | 1050 | 1051 | 1052 | SRC_ROI_8 |
| 2 | 80728470 | minus | 1053 | 1054 | 1055 | 1056 | CTNNA2_ROI_17 |
| 7 | 151499189 | minus | 1057 | 1058 | 1059 | 1060 | MLL3_ROI_39 |
| 6 | 3023129 | minus | 1061 | 1062 | 1063 | 1064 | RIPK1_ROI_2 |
| 3 | 41241299 | plus | 1065 | 1066 | 1067 | 1068 | CTNNB1_ROI_3 |
| 19 | 10324452 | plus | 1069 | 1070 | 1071 | 1072 | TYK2_ROI_15 |
| 1 | 11097989 | plus | 1073 | 1074 | 1075 | 1076 | FRAP1_ROI_48 |
| 10 | 55391619 | minus | 1077 | 1078 | 1079 | 1080 | PCDH15_ROI_21 |
| 6 | 69842612 | minus | 1081 | 1082 | 1083 | 1084 | BAI3_ROI_15 |
| 17 | 26686136 | minus | 1085 | 1086 | 1087 | 1088 | NF1_ROI_40 |
| 19 | 1014393 | plus | 1089 | 1090 | 1091 | 1092 | ABCA7_ROI_31 |
| 12 | 76886518 | minus | 1093 | 1094 | 1095 | 1096 | NAV3_ROI_5 |
| 4 | 107376846 | plus | 1097 | 1098 | 1099 | 1100 | MGC16169_ROI_11 |
| 7 | 140147531 | plus | 1101 | 1102 | 1103 | 1104 | BRAF_ROI_6 |
| 23 | 69585338 | plus | 1105 | 1106 | 1107 | 1108 | DLG3_ROI_2 |
| 20 | 61808645 | plus | 1109 | 1110 | 1111 | 1112 | ARFRP1_ROI_1 |
| 20 | 29596459 | minus | 1113 | 1114 | 1115 | 1116 | HM13_ROI_4 |
| 13 | 31811217 | plus | 1117 | 1118 | 1119 | 1120 | BRCA2_ROI_9 |
| 17 | 26708193 | minus | 1121 | 1122 | 1123 | 1124 | NF1_ROI_53 |
| 1 | 64397102 | plus | 1125 | 1126 | 1127 | 1128 | ROR1_ROI_8 |
| 18 | 20923277 | plus | 1129 | 1130 | 1131 | 1132 | ZNF521_ROI_6 |
| 12 | 130168789 | minus | 1133 | 1134 | 1135 | 1136 | GPR133_ROI_18 |
| 18 | 46858794 | minus | 1137 | 1138 | 1139 | 1140 | SMAD4_ROI_10 |
| 7 | 148160509 | plus | 1141 | 1142 | 1143 | 1144 | EZH2_ROI_3 |
| 19 | 10334291 | minus | 1145 | 1146 | 1147 | 1148 | TYK2_ROI_6 |
| 12 | 130186385 | plus | 1149 | 1150 | 1151 | 1152 | GPR133_ROI_21 |
| 22 | 28362823 | minus | 1153 | 1154 | 1155 | 1156 | NF2_ROI_2 |
| 15 | 20479420 | plus | 1157 | 1158 | 1159 | 1160 | CYFIP1_ROI_2 |
| 7 | 151511132 | minus | 1161 | 1162 | 1163 | 1164 | MLL3_ROI_34 |
| 1 | 11230696 | minus | 1165 | 1166 | 1167 | 1168 | FRAP1_ROI_6 |
| 8 | 113306026 | plus | 1169 | 1170 | 1171 | 1172 | CSMD3_ROI_72 |
| 12 | 119918298 | plus | 1173 | 1174 | 1175 | 1176 | HNF1A_ROI_5 |
| 13 | 31810046 | minus | 1177 | 1178 | 1179 | 1180 | BRCA2_ROI_9 |
| 5 | 24527639 | minus | 1181 | 1182 | 1183 | 1184 | CDH10_ROI_10 |
| 17 | 26700268 | minus | 1185 | 1186 | 1187 | 1188 | NF1_ROI_49 |
| 17 | 26709723 | minus | 1189 | 1190 | 1191 | 1192 | NF1_ROI_55 |
| 12 | 130187321 | plus | 1193 | 1194 | 1195 | 1196 | GPR133_ROI_22 |
| 10 | 42921680 | plus | 1197 | 1198 | 1199 | 1200 | RET_ROI_5 |
| 7 | 98440554 | minus | 1201 | 1202 | 1203 | 1204 | TRRAP_ROI_63 |
| 16 | 67404719 | minus | 1205 | 1206 | 1207 | 1208 | CDH1_ROI_9 |
| 12 | 130022035 | minus | 1209 | 1210 | 1211 | 1212 | GPR133_ROI_3 |
| 23 | 85655898 | minus | 1213 | 1214 | 1215 | 1216 | DACH2_ROI_3 |
| 13 | 31791419 | minus | 1217 | 1218 | 1219 | 1220 | BRCA2_ROI_2 |
| 1 | 74607813 | minus | 1221 | 1222 | 1223 | 1224 | TNNI3K_ROI_14 |
| 15 | 20544316 | plus | 1225 | 1226 | 1227 | 1228 | CYFIP1_ROI_23 |
| 19 | 11029801 | plus | 1229 | 1230 | 1231 | 1232 | SMARCA4_ROI_30 |
| 1 | 115052754 | minus | 1233 | 1234 | 1235 | 1236 | NRAS_ROI_4 |
| 3 | 132363792 | plus | 1237 | 1238 | 1239 | 1240 | NEK11_ROI_8 |
| 18 | 49267015 | plus | 1241 | 1242 | 1243 | 1244 | DCC_ROI_26 |
| 1 | 11109744 | minus | 1245 | 1246 | 1247 | 1248 | FRAP1_ROI_42 |
| 17 | 26506977 | plus | 1249 | 1250 | 1251 | 1252 | NF1_ROI_2 |
| 5 | 112203353 | minus | 1253 | 1254 | 1255 | 1256 | APC_ROI_15 |
| 11 | 107707231 | plus | 1257 | 1258 | 1259 | 1260 | ATM_ROI_48 |
| 7 | 98417178 | plus | 1261 | 1262 | 1263 | 1264 | TRRAP_ROI_53 |
| 18 | 32515347 | minus | 1265 | 1266 | 1267 | 1268 | FHOD3_ROI_12 |
| 11 | 107695930 | minus | 1269 | 1270 | 1271 | 1272 | ATM_ROI_41 |
| 19 | 10990654 | minus | 1273 | 1274 | 1275 | 1276 | SMARCA4_ROI_16 |
| 2 | 179356542 | plus | 1277 | 1278 | 1279 | 1280 | TTN_ROI_15 |
| 1 | 11239670 | plus | 1281 | 1282 | 1283 | 1284 | FRAP1_ROI_3 |
| 7 | 140100425 | plus | 1285 | 1286 | 1287 | 1288 | BRAF_ROI_14 |
| 18 | 57318669 | minus | 1289 | 1290 | 1291 | 1292 | CDH20_ROI_3 |
| 20 | 29596261 | plus | 1293 | 1294 | 1295 | 1296 | HM13_ROI_4 |
| 16 | 86302192 | plus | 1297 | 1298 | 1299 | 1300 | KLHDC4_ROI_9 |
| 1 | 11127352 | minus | 1301 | 1302 | 1303 | 1304 | FRAP1_ROI_32 |
| 15 | 20551387 | plus | 1305 | 1306 | 1307 | 1308 | CYFIP1_ROI_27 |
| 7 | 81450528 | minus | 1309 | 1310 | 1311 | 1312 | CACNA2D1_ROI_23 |
| 15 | 20496331 | plus | 1313 | 1314 | 1315 | 1316 | CYFIP1_ROI_9 |
| 7 | 81531406 | plus | 1317 | 1318 | 1319 | 1320 | CACNA2D1_ROI_9 |
| 11 | 69165113 | plus | 1321 | 1322 | 1323 | 1324 | CCND1_ROI_1 |
| 2 | 179377566 | minus | 1325 | 1326 | 1327 | 1328 | TTN_ROI_1 |
| 7 | 113305967 | plus | 1329 | 1330 | 1331 | 1332 | PPP1R3A_ROI_3 |
| 1 | 115060096 | plus | 1333 | 1334 | 1335 | 1336 | NRAS_ROI_1 |
| 12 | 119915696 | plus | 1337 | 1338 | 1339 | 1340 | HNF1A_ROI_3 |
| 2 | 80670072 | minus | 1341 | 1342 | 1343 | 1344 | CTNNA2_ROI_14 |
| 7 | 148175057 | plus | 1345 | 1346 | 1347 | 1348 | EZH2_ROI_1 |
| 22 | 28384028 | plus | 1349 | 1350 | 1351 | 1352 | NF2_ROI_7 |
| 11 | 85639014 | minus | 1353 | 1354 | 1355 | 1356 | EED_ROI_2 |
| 5 | 14534677 | minus | 1357 | 1358 | 1359 | 1360 | TRIO_ROI_44 |
| 3 | 132551250 | minus | 1361 | 1362 | 1363 | 1364 | NEK11_ROI_15 |
| 7 | 148154607 | minus | 1365 | 1366 | 1367 | 1368 | EZH2_ROI_7 |
| 15 | 20551586 | minus | 1369 | 1370 | 1371 | 1372 | CYFIP1_ROI_27 |
| 19 | 7882884 | plus | 1373 | 1374 | 1375 | 1376 | MAP2K7_ROI_8 |
| 2 | 179350505 | minus | 1377 | 1378 | 1379 | 1380 | TTN_ROI_24 |
| 7 | 151510170 | minus | 1381 | 1382 | 1383 | 1384 | MLL3_ROI_35 |
| 15 | 20477147 | minus | 1385 | 1386 | 1387 | 1388 | CYFIP1_ROI_1 |
| 8 | 37810214 | plus | 1389 | 1390 | 1391 | 1392 | GPR124_ROI_10 |
| 3 | 89544938 | minus | 1393 | 1394 | 1395 | 1396 | EPHA3_ROI_10 |
| 18 | 32428598 | plus | 1397 | 1398 | 1399 | 1400 | FHOD3_ROI_7 |
| 4 | 1773252 | minus | 1401 | 1402 | 1403 | 1404 | FGFR3_ROI_4 |
| 19 | 10991137 | plus | 1405 | 1406 | 1407 | 1408 | SMARCA4_ROI_17 |
| 5 | 14534193 | plus | 1409 | 1410 | 1411 | 1412 | TRIO_ROI_43 |
| 4 | 1777921 | plus | 1413 | 1414 | 1415 | 1416 | FGFR3_ROI_10 |
| 4 | 107435699 | minus | 1417 | 1418 | 1419 | 1420 | MGC16169_ROI_2 |
| 9 | 21958177 | plus | 1421 | 1422 | 1423 | 1424 | CDKN2A_ROI_4 |
| 8 | 113315829 | minus | 1425 | 1426 | 1427 | 1428 | CSMD3_ROI_69 |
| 3 | 36755101 | minus | 1429 | 1430 | 1431 | 1432 | DCLK3_ROI_1 |
| 14 | 102539967 | plus | 1433 | 1434 | 1435 | 1436 | CDC42BPB_ROI_4 |
| 18 | 49102472 | minus | 1437 | 1438 | 1439 | 1440 | DCC_ROI_14 |
| 11 | 64330363 | minus | 1441 | 1442 | 1443 | 1444 | MEN1_ROI_5 |
| 1 | 74606037 | minus | 1445 | 1446 | 1447 | 1448 | TNNI3K_ROI_12 |
| 1 | 115052549 | plus | 1449 | 1450 | 1451 | 1452 | NRAS_ROI_4 |
| 15 | 20520454 | plus | 1453 | 1454 | 1455 | 1456 | CYFIP1_ROI_19 |
| 10 | 42926511 | plus | 1457 | 1458 | 1459 | 1460 | RET_ROI_7 |
| 19 | 14936119 | minus | 1461 | 1462 | 1463 | 1464 | SLC1A6_ROI_4 |
| 15 | 20498473 | minus | 1465 | 1466 | 1467 | 1468 | CYFIP1_ROI_10 |
| 5 | 112205714 | minus | 1469 | 1470 | 1471 | 1472 | APC_ROI_15 |
| 18 | 32552476 | minus | 1473 | 1474 | 1475 | 1476 | FHOD3_ROI_16 |
| 19 | 14940264 | minus | 1477 | 1478 | 1479 | 1480 | SLC1A6_ROI_3 |
| 15 | 20554187 | plus | 1481 | 1482 | 1483 | 1484 | CYFIP1_ROI_28 |
| 19 | 14943631 | minus | 1485 | 1486 | 1487 | 1488 | SLC1A6_ROI_2 |
| 7 | 98393449 | minus | 1489 | 1490 | 1491 | 1492 | TRRAP_ROI_38 |
| 1 | 11115766 | minus | 1493 | 1494 | 1495 | 1496 | FRAP1_ROI_35 |
| 2 | 179340824 | minus | 1497 | 1498 | 1499 | 1500 | TTN_ROI_33 |
| 4 | 107335174 | plus | 1501 | 1502 | 1503 | 1504 | MGC16169_ROI_18 |
| 8 | 113315623 | minus | 1505 | 1506 | 1507 | 1508 | CSMD3_ROI_69 |
| 18 | 51168953 | plus | 1509 | 1510 | 1511 | 1512 | TCF4_ROI_6 |
| 2 | 197972874 | plus | 1513 | 1514 | 1515 | 1516 | SF3B1_ROI_17 |
| 1 | 6104065 | minus | 1517 | 1518 | 1519 | 1520 | CHD5_ROI_29 |
| 3 | 89342303 | minus | 1521 | 1522 | 1523 | 1524 | EPHA3_ROI_3 |
| 18 | 41925496 | plus | 1525 | 1526 | 1527 | 1528 | ATP5A1_ROI_3 |
| 2 | 1467225 | plus | 1529 | 1530 | 1531 | 1532 | TPO_ROI_8 |
| 17 | 35137269 | minus | 1533 | 1534 | 1535 | 1536 | ERBB2_ROI_23 |
| 1 | 150591471 | minus | 1537 | 1538 | 1539 | 1540 | FLG2_ROI_2 |
| 19 | 10333117 | minus | 1541 | 1542 | 1543 | 1544 | TYK2_ROI_8 |
| 1 | 74608410 | plus | 1545 | 1546 | 1547 | 1548 | TNNI3K_ROI_15 |
| 14 | 102486426 | plus | 1549 | 1550 | 1551 | 1552 | CDC42BPB_ROI_24 |
| 1 | 11127142 | plus | 1553 | 1554 | 1555 | 1556 | FRAP1_ROI_32 |
| 12 | 76924180 | plus | 1557 | 1558 | 1559 | 1560 | NAV3_ROI_8 |
| 6 | 3022903 | minus | 1561 | 1562 | 1563 | 1564 | RIPK1_ROI_2 |
| 22 | 28380595 | plus | 1565 | 1566 | 1567 | 1568 | NF2_ROI_5 |
| 1 | 11090085 | minus | 1569 | 1570 | 1571 | 1572 | FRAP1_ROI_55 |
| 6 | 80777822 | minus | 1573 | 1574 | 1575 | 1576 | TTK_ROI_5 |
| 7 | 151467054 | plus | 1577 | 1578 | 1579 | 1580 | MLL3_ROI_56 |
| 7 | 98388576 | plus | 1581 | 1582 | 1583 | 1584 | TRRAP_ROI_35 |
| 19 | 14943419 | plus | 1585 | 1586 | 1587 | 1588 | SLC1A6_ROI_2 |
| 13 | 31818814 | plus | 1589 | 1590 | 1591 | 1592 | BRCA2_ROI_11 |
| 2 | 79954787 | minus | 1593 | 1594 | 1595 | 1596 | CTNNA2_ROI_5 |
| 7 | 98316560 | plus | 1597 | 1598 | 1599 | 1600 | TRRAP_ROI_1 |
| 18 | 43650694 | plus | 1601 | 1602 | 1603 | 1604 | SMAD2_ROI_2 |
| 10 | 55368431 | plus | 1605 | 1606 | 1607 | 1608 | PCDH15_ROI_24 |
| 22 | 28408859 | plus | 1609 | 1610 | 1611 | 1612 | NF2_ROI_16 |
| 2 | 197973321 | minus | 1613 | 1614 | 1615 | 1616 | SF3B1_ROI_17 |
| 19 | 11002256 | plus | 1617 | 1618 | 1619 | 1620 | SMARCA4_ROI_24 |
| 7 | 151477108 | minus | 1621 | 1622 | 1623 | 1624 | MLL3_ROI_51 |
| 17 | 10381125 | plus | 1625 | 1626 | 1627 | 1628 | MYH2_ROI_10 |
| 17 | 26583963 | minus | 1629 | 1630 | 1631 | 1632 | NF1_ROI_26 |
| 8 | 113416808 | minus | 1633 | 1634 | 1635 | 1636 | CSMD3_ROI_46 |
| 1 | 173573323 | minus | 1637 | 1638 | 1639 | 1640 | TNR_ROI_17 |
| 1 | 11225881 | minus | 1641 | 1642 | 1643 | 1644 | FRAP1_ROI_7 |
| 8 | 114036082 | minus | 1645 | 1646 | 1647 | 1648 | CSMD3_ROI_9 |
| 17 | 26711481 | plus | 1649 | 1650 | 1651 | 1652 | NF1_ROI_57 |
| 2 | 47871703 | minus | 1653 | 1654 | 1655 | 1656 | MSH6_ROI_2 |
| 8 | 113726546 | minus | 1657 | 1658 | 1659 | 1660 | CSMD3_ROI_21 |
| 1 | 11236444 | minus | 1661 | 1662 | 1663 | 1664 | FRAP1_ROI_5 |
| 5 | 24573476 | minus | 1665 | 1666 | 1667 | 1668 | CDH10_ROI_2 |
| 12 | 76939708 | minus | 1669 | 1670 | 1671 | 1672 | NAV3_ROI_9 |
| 17 | 27344847 | plus | 1673 | 1674 | 1675 | 1676 | SUZ12_ROI_11 |
| 8 | 114100420 | minus | 1677 | 1678 | 1679 | 1680 | CSMD3_ROI_7 |
| 7 | 148175258 | minus | 1681 | 1682 | 1683 | 1684 | EZH2_ROI_1 |
| 2 | 197969232 | minus | 1685 | 1686 | 1687 | 1688 | SF3B1_ROI_20 |
| 11 | 107707428 | minus | 1689 | 1690 | 1691 | 1692 | ATM_ROI_48 |
| 3 | 10158382 | plus | 1693 | 1694 | 1695 | 1696 | VHL_ROI_1 |
| 7 | 55236476 | minus | 1697 | 1698 | 1699 | 1700 | EGFR_ROI_26 |
| 8 | 37807513 | minus | 1701 | 1702 | 1703 | 1704 | GPR124_ROI_7 |
| 7 | 98388926 | minus | 1705 | 1706 | 1707 | 1708 | TRRAP_ROI_35 |
| 6 | 69722583 | minus | 1709 | 1710 | 1711 | 1712 | BAI3_ROI_5 |
| 3 | 180404835 | plus | 1713 | 1714 | 1715 | 1716 | PIK3CA_ROI_5 |
| 19 | 997079 | plus | 1717 | 1718 | 1719 | 1720 | ABCA7_ROI_9 |
| 6 | 3049308 | plus | 1721 | 1722 | 1723 | 1724 | RIPK1_ROI_7 |
| 17 | 26707974 | plus | 1725 | 1726 | 1727 | 1728 | NF1_ROI_53 |
| 19 | 7880798 | plus | 1729 | 1730 | 1731 | 1732 | MAP2K7_ROI_3 |
| 1 | 6138197 | minus | 1733 | 1734 | 1735 | 1736 | CHD5_ROI_4 |
| 12 | 77107745 | plus | 1737 | 1738 | 1739 | 1740 | NAV3_ROI_33 |
| 2 | 1405674 | plus | 1741 | 1742 | 1743 | 1744 | TPO_ROI_2 |
| 17 | 26586978 | minus | 1745 | 1746 | 1747 | 1748 | NF1_ROI_29 |
| 14 | 102479831 | plus | 1749 | 1750 | 1751 | 1752 | CDC42BPB_ROI_29 |
| 5 | 112179021 | minus | 1753 | 1754 | 1755 | 1756 | APC_ROI_8 |
| 1 | 11099597 | plus | 1757 | 1758 | 1759 | 1760 | FRAP1_ROI_47 |
| 19 | 14928434 | minus | 1761 | 1762 | 1763 | 1764 | SLC1A6_ROI_6 |
| 8 | 114057092 | plus | 1765 | 1766 | 1767 | 1768 | CSMD3_ROI_8 |
| 7 | 98347703 | minus | 1769 | 1770 | 1771 | 1772 | TRRAP_ROI_16 |
| 17 | 35129416 | plus | 1773 | 1774 | 1775 | 1776 | ERBB2_ROI_14 |
| 18 | 51088122 | minus | 1777 | 1778 | 1779 | 1780 | TCF4_ROI_10 |
| 7 | 98336132 | plus | 1781 | 1782 | 1783 | 1784 | TRRAP_ROI_10 |
| 18 | 48843523 | plus | 1785 | 1786 | 1787 | 1788 | DCC_ROI_6 |
| 4 | 107391014 | minus | 1789 | 1790 | 1791 | 1792 | MGC16169_ROI_4 |
| 15 | 20496531 | minus | 1793 | 1794 | 1795 | 1796 | CYFIP1_ROI_9 |
| 20 | 29600343 | plus | 1797 | 1798 | 1799 | 1800 | HM13_ROI_5 |
| 19 | 35004385 | minus | 1801 | 1802 | 1803 | 1804 | CCNE1_ROI_7 |
| 18 | 41921923 | plus | 1805 | 1806 | 1807 | 1808 | ATP5A1_ROI_5 |
| 7 | 81437897 | minus | 1809 | 1810 | 1811 | 1812 | CACNA2D1_ROI_27 |
| 8 | 113416584 | plus | 1813 | 1814 | 1815 | 1816 | CSMD3_ROI_46 |
| 18 | 41928966 | plus | 1817 | 1818 | 1819 | 1820 | ATP5A1_ROI_2 |
| 7 | 148160703 | minus | 1821 | 1822 | 1823 | 1824 | EZH2_ROI_3 |
| 18 | 20923470 | minus | 1825 | 1826 | 1827 | 1828 | ZNF521_ROI_6 |
| 6 | 41665436 | plus | 1829 | 1830 | 1831 | 1832 | FOXP4_ROI_9 |
| 7 | 151495042 | minus | 1833 | 1834 | 1835 | 1836 | MLL3_ROI_41 |
| 7 | 98368670 | plus | 1837 | 1838 | 1839 | 1840 | TRRAP_ROI_25 |
| 2 | 79938497 | plus | 1841 | 1842 | 1843 | 1844 | CTNNA2_ROI_3 |
| 18 | 48959180 | plus | 1845 | 1846 | 1847 | 1848 | DCC_ROI_9 |
| 6 | 3030568 | minus | 1849 | 1850 | 1851 | 1852 | RIPK1_ROI_5 |
| 7 | 98370988 | plus | 1853 | 1854 | 1855 | 1856 | TRRAP_ROI_26 |
| 1 | 64247415 | plus | 1857 | 1858 | 1859 | 1860 | ROR1_ROI_2 |
| 2 | 79824871 | plus | 1861 | 1862 | 1863 | 1864 | CTNNA2_ROI_2 |
| 7 | 128630340 | minus | 1865 | 1866 | 1867 | 1868 | SMO_ROI_2 |
| 23 | 85856143 | minus | 1869 | 1870 | 1871 | 1872 | DACH2_ROI_6 |
| 3 | 49873941 | minus | 1873 | 1874 | 1875 | 1876 | CAMKV_ROI_4 |
| 1 | 11239427 | plus | 1877 | 1878 | 1879 | 1880 | FRAP1_ROI_3 |
| 7 | 81479421 | minus | 1881 | 1882 | 1883 | 1884 | CACNA2D1_ROI_15 |
| 8 | 113417907 | plus | 1885 | 1886 | 1887 | 1888 | CSMD3_ROI_45 |
| 17 | 11983731 | plus | 1889 | 1890 | 1891 | 1892 | MAP2K4_ROI_10 |
| 23 | 69631833 | plus | 1893 | 1894 | 1895 | 1896 | DLG3_ROI_15 |
| 23 | 70504023 | plus | 1897 | 1898 | 1899 | 1900 | TAF1_ROI_2 |
| 7 | 98353085 | minus | 1901 | 1902 | 1903 | 1904 | TRRAP_ROI_18 |
| 3 | 180410107 | minus | 1905 | 1906 | 1907 | 1908 | PIK3CA_ROI_6 |
| 8 | 114359847 | plus | 1909 | 1910 | 1911 | 1912 | CSMD3_ROI_4 |
| 1 | 11112232 | plus | 1913 | 1914 | 1915 | 1916 | FRAP1_ROI_37 |
| 8 | 113654756 | plus | 1917 | 1918 | 1919 | 1920 | CSMD3_ROI_25 |
| 2 | 79954560 | plus | 1921 | 1922 | 1923 | 1924 | CTNNA2_ROI_5 |
| 5 | 14427278 | minus | 1925 | 1926 | 1927 | 1928 | TRIO_ROI_19 |
| 6 | 69710445 | minus | 1929 | 1930 | 1931 | 1932 | BAI3_ROI_4 |
| 7 | 81449768 | plus | 1933 | 1934 | 1935 | 1936 | CACNA2D1_ROI_24 |
| 11 | 107628773 | minus | 1937 | 1938 | 1939 | 1940 | ATM_ROI_10 |
| 7 | 140124267 | minus | 1941 | 1942 | 1943 | 1944 | BRAF_ROI_12 |
| 10 | 55368644 | minus | 1945 | 1946 | 1947 | 1948 | PCDH15_ROI_24 |
| 2 | 197971473 | minus | 1949 | 1950 | 1951 | 1952 | SF3B1_ROI_18 |
| 12 | 50663900 | plus | 1953 | 1954 | 1955 | 1956 | ACVR1B_ROI_5 |
| 10 | 42920569 | minus | 1957 | 1958 | 1959 | 1960 | RET_ROI_4 |
| 12 | 25271285 | plus | 1961 | 1962 | 1963 | 1964 | KRAS_ROI_2 |
| 3 | 132335313 | plus | 1965 | 1966 | 1967 | 1968 | NEK11_ROI_5 |
| 4 | 107392376 | minus | 1969 | 1970 | 1971 | 1972 | MGC16169_ROI_3 |
| 5 | 112191375 | plus | 1973 | 1974 | 1975 | 1976 | APC_ROI_12 |
| 6 | 70005740 | minus | 1977 | 1978 | 1979 | 1980 | BAI3_ROI_18 |
| 18 | 48959405 | minus | 1981 | 1982 | 1983 | 1984 | DCC_ROI_9 |
| 2 | 179348985 | minus | 1985 | 1986 | 1987 | 1988 | TTN_ROI_25 |
| 3 | 41240871 | plus | 1989 | 1990 | 1991 | 1992 | CTNNB1_ROI_2 |
| 7 | 151523874 | plus | 1993 | 1994 | 1995 | 1996 | MLL3_ROI_28 |
| 18 | 46847475 | minus | 1997 | 1998 | 1999 | 2000 | SMAD4_ROI_8 |
| 17 | 26691499 | plus | 2001 | 2002 | 2003 | 2004 | NF1_ROI_47 |
| 13 | 31811428 | plus | 2005 | 2006 | 2007 | 2008 | BRCA2_ROI_9 |
| 12 | 77118234 | plus | 2009 | 2010 | 2011 | 2012 | NAV3_ROI_37 |
| 10 | 55370660 | minus | 2013 | 2014 | 2015 | 2016 | PCDH15_ROI_23 |
| 22 | 28362590 | plus | 2017 | 2018 | 2019 | 2020 | NF2_ROI_2 |
| 11 | 64330130 | plus | 2021 | 2022 | 2023 | 2024 | MEN1_ROI_5 |
| 15 | 20492205 | minus | 2025 | 2026 | 2027 | 2028 | CYFIP1_ROI_8 |
| 1 | 11238642 | minus | 2029 | 2030 | 2031 | 2032 | FRAP1_ROI_4 |
| 7 | 128632130 | plus | 2033 | 2034 | 2035 | 2036 | SMO_ROI_3 |
| 18 | 21059225 | minus | 2037 | 2038 | 2039 | 2040 | ZNF521_ROI_3 |
| 9 | 93533046 | minus | 2041 | 2042 | 2043 | 2044 | ROR2_ROI_7 |
| 23 | 47307518 | plus | 2045 | 2046 | 2047 | 2048 | ARAF_ROI_2 |
| 7 | 81451882 | minus | 2049 | 2050 | 2051 | 2052 | CACNA2D1_ROI_22 |
| 18 | 48843745 | minus | 2053 | 2054 | 2055 | 2056 | DCC_ROI_6 |
| 20 | 61803515 | minus | 2057 | 2058 | 2059 | 2060 | ARFRP1_ROI_5 |
| 6 | 80804253 | plus | 2061 | 2062 | 2063 | 2064 | TTK_ROI_16 |
| 2 | 80473697 | plus | 2065 | 2066 | 2067 | 2068 | CTNNA2_ROI_7 |
| 3 | 41252256 | minus | 2069 | 2070 | 2071 | 2072 | CTNNB1_ROI_8 |
| 23 | 69637323 | plus | 2073 | 2074 | 2075 | 2076 | DLG3_ROI_21 |
| 18 | 21059954 | minus | 2077 | 2078 | 2079 | 2080 | ZNF521_ROI_3 |
| 1 | 173602817 | plus | 2081 | 2082 | 2083 | 2084 | TNR_ROI_8 |
| 8 | 113940400 | plus | 2085 | 2086 | 2087 | 2088 | CSMD3_ROI_12 |
| 10 | 42920256 | plus | 2089 | 2090 | 2091 | 2092 | RET_ROI_4 |
| 5 | 112144236 | plus | 2093 | 2094 | 2095 | 2096 | APC_ROI_5 |
| 10 | 89707440 | plus | 2097 | 2098 | 2099 | 2100 | PTEN_ROI_7 |
| 18 | 41931986 | plus | 2101 | 2102 | 2103 | 2104 | ATP5A1_ROI_1 |
| 2 | 179346305 | minus | 2105 | 2106 | 2107 | 2108 | TTN_ROI_28 |
| 11 | 107708733 | minus | 2109 | 2110 | 2111 | 2112 | ATM_ROI_50 |
| 23 | 85290131 | plus | 2113 | 2114 | 2115 | 2116 | DACH2_ROI_1 |
| 3 | 180401622 | plus | 2117 | 2118 | 2119 | 2120 | PIK3CA_ROI_3 |
| 20 | 35455582 | plus | 2121 | 2122 | 2123 | 2124 | SRC_ROI_3 |
| 18 | 32576663 | minus | 2125 | 2126 | 2127 | 2128 | FHOD3_ROI_19 |
| 1 | 11198675 | minus | 2129 | 2130 | 2131 | 2132 | FRAP1_ROI_18 |
| 8 | 37818947 | minus | 2133 | 2134 | 2135 | 2136 | GPR124_ROI_19 |
| 7 | 98412348 | plus | 2137 | 2138 | 2139 | 2140 | TRRAP_ROI_50 |
| 11 | 64330909 | plus | 2141 | 2142 | 2143 | 2144 | MEN1_ROI_4 |
| 1 | 74677809 | minus | 2145 | 2146 | 2147 | 2148 | TNNI3K_ROI_18 |
| 7 | 148137330 | minus | 2149 | 2150 | 2151 | 2152 | EZH2_ROI_17 |
| 7 | 55236884 | minus | 2153 | 2154 | 2155 | 2156 | EGFR_ROI_27 |
| 1 | 173560193 | minus | 2157 | 2158 | 2159 | 2160 | TNR_ROI_20 |
| 12 | 130041431 | minus | 2161 | 2162 | 2163 | 2164 | GPR133_ROI_6 |
| 23 | 70246761 | minus | 2165 | 2166 | 2167 | 2168 | IL2RG_ROI_4 |
| 16 | 23614616 | minus | 2169 | 2170 | 2171 | 2172 | ERN2_ROI_13 |
| 18 | 49267211 | minus | 2173 | 2174 | 2175 | 2176 | DCC_ROI_26 |
| 1 | 11111110 | minus | 2177 | 2178 | 2179 | 2180 | FRAP1_ROI_39 |
| 18 | 46847237 | plus | 2181 | 2182 | 2183 | 2184 | SMAD4_ROI_8 |
| 16 | 86321665 | minus | 2185 | 2186 | 2187 | 2188 | KLHDC4_ROI_6 |
| 1 | 74591546 | minus | 2189 | 2190 | 2191 | 2192 | TNNI3K_ROI_9 |
| 2 | 179355950 | minus | 2193 | 2194 | 2195 | 2196 | TTN_ROI_16 |
| 6 | 80806482 | plus | 2197 | 2198 | 2199 | 2200 | TTK_ROI_18 |
| 3 | 132366810 | plus | 2201 | 2202 | 2203 | 2204 | NEK11_ROI_9 |
| 12 | 76886280 | plus | 2205 | 2206 | 2207 | 2208 | NAV3_ROI_5 |
| 1 | 11095346 | plus | 2209 | 2210 | 2211 | 2212 | FRAP1_ROI_51 |
| 2 | 1436343 | plus | 2213 | 2214 | 2215 | 2216 | TPO_ROI_5 |
| 3 | 41253051 | plus | 2217 | 2218 | 2219 | 2220 | CTNNB1_ROI_9 |
| 2 | 1476451 | plus | 2221 | 2222 | 2223 | 2224 | TPO_ROI_10 |
| 17 | 11954366 | plus | 2225 | 2226 | 2227 | 2228 | MAP2K4_ROI_6 |
| 8 | 113310011 | plus | 2229 | 2230 | 2231 | 2232 | CSMD3_ROI_71 |
| 1 | 11109504 | plus | 2233 | 2234 | 2235 | 2236 | FRAP1_ROI_42 |
| 1 | 74605378 | plus | 2237 | 2238 | 2239 | 2240 | TNNI3K_ROI_11 |
| 23 | 70559641 | plus | 2241 | 2242 | 2243 | 2244 | TAF1_ROI_29 |
| 18 | 32335743 | plus | 2245 | 2246 | 2247 | 2248 | FHOD3_ROI_4 |
| 23 | 70247537 | minus | 2249 | 2250 | 2251 | 2252 | IL2RG_ROI_2 |
| 12 | 50664129 | minus | 2253 | 2254 | 2255 | 2256 | ACVR1B_ROI_5 |
| 1 | 6088892 | plus | 2257 | 2258 | 2259 | 2260 | CHD5_ROI_37 |
| 1 | 64378230 | plus | 2261 | 2262 | 2263 | 2264 | ROR1_ROI_6 |
| 3 | 89539856 | plus | 2265 | 2266 | 2267 | 2268 | EPHA3_ROI_9 |
| 10 | 89643757 | minus | 2269 | 2270 | 2271 | 2272 | PTEN_ROI_2 |
| 3 | 180410518 | plus | 2273 | 2274 | 2275 | 2276 | PIK3CA_ROI_7 |
| 12 | 50673985 | plus | 2277 | 2278 | 2279 | 2280 | ACVR1B_ROI_9 |
| 1 | 11193308 | plus | 2281 | 2282 | 2283 | 2284 | FRAP1_ROI_22 |
| 2 | 179340581 | plus | 2285 | 2286 | 2287 | 2288 | TTN_ROI_33 |
| 1 | 74487581 | plus | 2289 | 2290 | 2291 | 2292 | TNNI3K_ROI_3 |
| 1 | 56934290 | minus | 2293 | 2294 | 2295 | 2296 | PRKAA2_ROI_6 |
| 2 | 197973813 | minus | 2297 | 2298 | 2299 | 2300 | SF3B1_ROI_16 |
| 17 | 26688932 | minus | 2301 | 2302 | 2303 | 2304 | NF1_ROI_44 |
| 19 | 1005248 | minus | 2305 | 2306 | 2307 | 2308 | ABCA7_ROI_20 |
| 19 | 34995286 | plus | 2309 | 2310 | 2311 | 2312 | CCNE1_ROI_2 |
| 2 | 148400008 | minus | 2313 | 2314 | 2315 | 2316 | ACVR2A_ROI_10 |
| 18 | 49171873 | plus | 2317 | 2318 | 2319 | 2320 | DCC_ROI_17 |
| 7 | 55178342 | plus | 2321 | 2322 | 2323 | 2324 | EGFR_ROI_3 |
| 7 | 98414370 | minus | 2325 | 2326 | 2327 | 2328 | TRRAP_ROI_52 |
| 15 | 20479700 | plus | 2329 | 2330 | 2331 | 2332 | CYFIP1_ROI_3 |
| 23 | 85290367 | minus | 2333 | 2334 | 2335 | 2336 | DACH2_ROI_1 |
| 23 | 69586693 | plus | 2337 | 2338 | 2339 | 2340 | DLG3_ROI_5 |
| 8 | 113881417 | plus | 2341 | 2342 | 2343 | 2344 | CSMD3_ROI_14 |
| 2 | 106789743 | minus | 2345 | 2346 | 2347 | 2348 | ST6GAL2_ROI_5 |
| 18 | 19028260 | plus | 2349 | 2350 | 2351 | 2352 | CABLES1_ROI_4 |
| 7 | 148137084 | plus | 2353 | 2354 | 2355 | 2356 | EZH2_ROI_18 |
| 23 | 122364535 | minus | 2357 | 2358 | 2359 | 2360 | GRIA3_ROI_8 |
| 19 | 11005347 | minus | 2361 | 2362 | 2363 | 2364 | SMARCA4_ROI_26 |
| 23 | 122426549 | minus | 2365 | 2366 | 2367 | 2368 | GRIA3_ROI_13 |
| 7 | 55226966 | minus | 2369 | 2370 | 2371 | 2372 | EGFR_ROI_22 |
| 1 | 58774623 | plus | 2373 | 2374 | 2375 | 2376 | OMA1_ROI_2 |
| 4 | 107373394 | plus | 2377 | 2378 | 2379 | 2380 | MGC16169_ROI_14 |
| 12 | 76858850 | minus | 2381 | 2382 | 2383 | 2384 | NAV3_ROI_3 |
| 5 | 112204035 | minus | 2385 | 2386 | 2387 | 2388 | APC_ROI_15 |
| 2 | 179355478 | minus | 2389 | 2390 | 2391 | 2392 | TTN_ROI_17 |
| 4 | 1773454 | minus | 2393 | 2394 | 2395 | 2396 | FGFR3_ROI_4 |
| 2 | 179364894 | plus | 2397 | 2398 | 2399 | 2400 | TTN_ROI_8 |
| 8 | 37810417 | minus | 2401 | 2402 | 2403 | 2404 | GPR124_ROI_10 |
| 2 | 80688784 | minus | 2405 | 2406 | 2407 | 2408 | CTNNA2_ROI_16 |
| 8 | 113328275 | plus | 2409 | 2410 | 2411 | 2412 | CSMD3_ROI_65 |
| 18 | 49215480 | minus | 2413 | 2414 | 2415 | 2416 | DCC_ROI_22 |
| 8 | 113432430 | plus | 2417 | 2418 | 2419 | 2420 | CSMD3_ROI_41 |
| 17 | 26709474 | plus | 2421 | 2422 | 2423 | 2424 | NF1_ROI_55 |
| 17 | 26507173 | minus | 2425 | 2426 | 2427 | 2428 | NF1_ROI_2 |
| 23 | 70596178 | minus | 2429 | 2430 | 2431 | 2432 | TAF1_ROI_34 |
| 5 | 14346046 | minus | 2433 | 2434 | 2435 | 2436 | TRIO_ROI_6 |
| 23 | 69638746 | minus | 2437 | 2438 | 2439 | 2440 | DLG3_ROI_22 |
| 18 | 51079664 | minus | 2441 | 2442 | 2443 | 2444 | TCF4_ROI_11 |
| 3 | 89239439 | plus | 2445 | 2446 | 2447 | 2448 | EPHA3_ROI_1 |
| 18 | 49177526 | plus | 2449 | 2450 | 2451 | 2452 | DCC_ROI_18 |
| 3 | 73522858 | minus | 2453 | 2454 | 2455 | 2456 | PDZRN3_ROI_6 |
| 12 | 77103362 | plus | 2457 | 2458 | 2459 | 2460 | NAV3_ROI_30 |
| 19 | 10996015 | minus | 2461 | 2462 | 2463 | 2464 | SMARCA4_ROI_20 |
| 10 | 55391368 | plus | 2465 | 2466 | 2467 | 2468 | PCDH15_ROI_21 |
| 7 | 148142888 | plus | 2469 | 2470 | 2471 | 2472 | EZH2_ROI_13 |
| 19 | 11002470 | minus | 2473 | 2474 | 2475 | 2476 | SMARCA4_ROI_24 |
| 7 | 55236225 | plus | 2477 | 2478 | 2479 | 2480 | EGFR_ROI_26 |
| 19 | 1005419 | plus | 2481 | 2482 | 2483 | 2484 | ABCA7_ROI_21 |
| 17 | 35136191 | plus | 2485 | 2486 | 2487 | 2488 | ERBB2_ROI_21 |
| 1 | 74574232 | plus | 2489 | 2490 | 2491 | 2492 | TNNI3K_ROI_7 |
| 1 | 74674202 | plus | 2493 | 2494 | 2495 | 2496 | TNNI3K_ROI_16 |
| 17 | 26616383 | minus | 2497 | 2498 | 2499 | 2500 | NF1_ROI_36 |
| 22 | 28400675 | plus | 2501 | 2502 | 2503 | 2504 | NF2_ROI_13 |
| 4 | 1776295 | minus | 2505 | 2506 | 2507 | 2508 | FGFR3_ROI_7 |
| 5 | 14560081 | plus | 2509 | 2510 | 2511 | 2512 | TRIO_ROI_55 |

The 5′ end and the 3′ end of the capture oliogonucleotides were blocked and did not contain phosphate or hydroxyl groups and 10 thymines were substituted with uracils to facilitate fragmentation and purification of the splint oligonucleotides after circularization. All oligonucleotides were synthesized at the Stanford Genome Technology Center (Stanford, Calif.). In an alternative design we substituted the central 40 bp of the capture oligonucleotide with a sequence comprising the Illlumina® sequencer adapter sequence. This has the advantage of creating amplicons ready for sequencing in a single amplification reaction, thus greatly facilitating the workflow. IIlumina® adapter sequences are available to anyone using their products; any approximately 35 bases, designed to allow attachment of the DNA to be sequenced to the surface of the flow cells used. Other sequencing systems would use other adapters.

### Targeted Genomic Circularization

High quality genomic DNA from flash-frozen tissues was first sonicated for 10 minutes in the Bioruptor to a size of 500-1000 bps. The hybridication reactions contained 0.5 μg dsDNA or 3-4 μg ddDNA and 50 pM of each of the capture oligonucleotides. After a brief denaturation step, the mixture was incubated in the PCR machine using a touchdown protocol ranging from 70-50° C. and 30-60 minutes for each step. Then a mixture of the cleavage enzymes (ExoI and Taq) and circularization enzyme (Ampligase or Taq ligase) were added to each tube and the reactions were incubated for 1 hour at 37° C. followed by a touchup protocol from 50-72° C. for 30 minute at each step. Excess oligonucleotides in the reactions were cleaved by uracil excision. After a brief purification using the Spin-20 columns, the captured DNA fragments were amplified using the high-fidelity Phusion polymerase and either the generic primer (e.g. ID 102) [9] or IIlumina PE-primers for 38-39 cycles. The PCR products were purified using the Fermentas kit.

### Sequencing Library Construction

The captured target DNA amplified with the generic PCR primers were ligated to PE-adapters after “A-tailing” and gel purified. They were then amplified for 10-12 cycles using the PE primers and re-purified from agarose gel. For DNA fragments captured with built-in PE primer sites, they were first purified away from the primer-dimers by gel electrophoresis and re-amplified for 5 cycles using the short PE primers. After quantitation by the SYBR based fluorescence assay, the libraries were sequenced on Illumina HiSeq or GAIIx using standard conditions.

### Sequencing

10 pM of PCR amplified library and 1.5 pM of circularized DNA were sequenced using the Illumina Genome Analyzer IIx. Circular library obtained from 1 μg of starting material was introduced to the sequencing experiment. After sample dilution using hybridization buffer, 20% of the prepared sample (representing 200 ng of starting material) was hybridized in the flow cell.

### Data Analysis

Sequence reads were aligned to the human genome version hg19 using ELAND software. The target regions were defined as the ranges from each target specific site to 41 bases upstream or downstream of it (depending on the orientation of the capture oligonucleotide). The interval of 41 bases was selected because the read length in these experiments was 42. In a paired-end experiment the target region contained both ends of the circularized fragments, while single-read sequencing targeted only 3′ ends of the circularized fragments. To assess the specificity of the capture, the numbers of sequence reads mapping inside and outside the target region were compared. To illustrate the uniformity of the assay, the reads that aligned perfectly with the specific capture sequences were counted. Read counts were then sorted and normalized using the median sequence yield value from each experiment. The genomic distance between the target specific sites indicates the circle size. In addition, guanine and cytosine proportions within the target sites were determined. The present capture oligonucleotide contains two target specific sites and each site was analyzed separately. To analyze the annealing properties during circularization-hybridization reaction, target specific sites within a single capture oligonucleotide as high or low G+C were classified. Circle sizes and G+C proportions with the sequence yields for each oligonucleotide were then plotted.

### Example 2

### Assessment of Overall Capture Coverage

In a proof of principle experiment, we used a set of previously described capture oligonucleotides [9]. Because we had determined that amplicon size was an important parameter for this type of selective circularization, we chose a subset of 628 capture oligonucleotides, each targeting a 150-250 base region. The assay targets a total of 123,982 bases. We compared the yield and the reproducibility of targeting reactions using DNA extracted from either fresh frozen tissue or FFPE blocks of three individuals. Both fresh frozen and FFPE samples are derived from normal colon according to the pathology reports.

The resulting capture amplicons from matched genomic DNA samples derived from either flash-frozen or FFPE material were concatenated using T4 DNA ligase and mechanically fragmented prior to library preparation. Replicate sequencing was conducted in triplicate to identify sequencing specific errors. The fragmented amplicons ligated to a 4-plex paired-end indexing adapters for two samples from individuals 751 and 761 [13]. The four libraries were combined and sequenced in three separate lanes of an Illlumina GAIIx sequencer. For matched samples from individual 780, paired end sequencing was conducted on both the flash tissue and FFPE derived material in separate full sized lanes. Sequence reads were aligned to the human genome reference. Given the replicate sequencing and matched samples, there were a total of 14 separate sequencing data sets. Each was analyzed separately (Table 1).

TABLE 1
Capture yield comparison
total bases targeted: 123982

| | | | Coverage greater than: | | | cov >= | | | lane |
|---|---|---|---|---|---|---|---|---|---|
| patient | sample | replicate | 1 | 10 | 20 | 10(%) | average | median | fraction |
| 751 | ffpe | rep1 | 109560 | 104038 | 100403 | 84 | 2513.8 | 368 | 0.25 |
| | | rep2 | 110086 | 104274 | 100193 | 84 | 2485.6 | 367 | 0.25 |
| | | rep3 | 109981 | 104458 | 100223 | 84 | 2555.4 | 373 | 0.25 |
| | fresh | rep1 | 115336 | 109041 | 104225 | 88 | 2251 | 439 | 0.25 |
| | | rep2 | 115330 | 108512 | 103312 | 88 | 2190.8 | 427 | 0.25 |
| | | rep3 | 115308 | 108457 | 103683 | 87 | 2217 | 432 | 0.25 |
| 761 | ffpe | rep1 | 103859 | 97964 | 94008 | 79 | 2613.7 | 288 | 0.25 |
| | | rep2 | 104590 | 97536 | 93888 | 79 | 2594.2 | 288 | 0.25 |
| | | rep3 | 104374 | 97666 | 94077 | 79 | 2672.2 | 296 | 0.25 |
| | fresh | rep1 | 118489 | 111115 | 106580 | 90 | 3107.7 | 613 | 0.25 |
| | | rep2 | 118553 | 110841 | 106306 | 89 | 3083.3 | 612 | 0.25 |
| | | rep3 | 118632 | 111387 | 106717 | 90 | 3167.4 | 627 | 0.25 |
| 780 | ffpe | rep1 | 110890 | 104523 | 102638 | 84 | 14712.2 | 1748 | 1 |
| | fresh | rep1 | 118687 | 113881 | 110414 | 92 | 3414.13 | 691 | 1 |

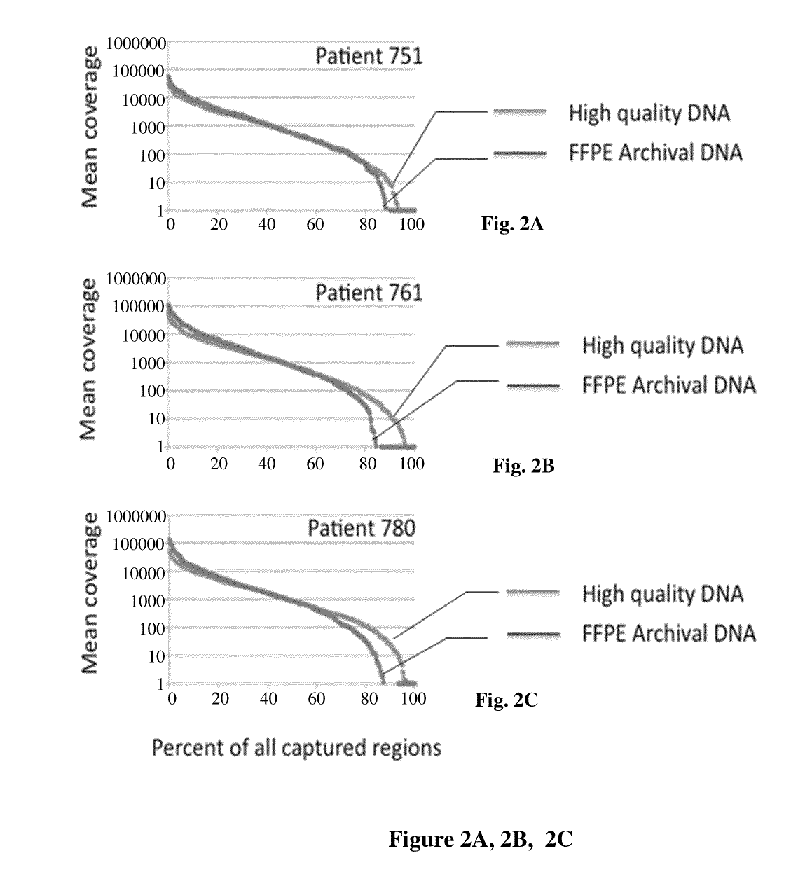

Overall, the sequence coverage is very reproducible among the replicates for each individual's samples. As noted in Table 1 the sequence coverage at 10× coverage ranges from 79% to 92% and is 5 to 10% lower for the FFPE derived than for the flash tissue derived samples. The uniformity of capture between the two types of starting material and for all three patient's DNA was compared (FIG. 2). Approximately 5-10% fewer regions are captured with a sequence coverage greater than 10× in FFPE relative to flash-frozen tissue.

It was determined that the sensitivity of detection of heterozygote SNVs in the targeted resequencing from FFPE versus flash-frozen derived DNA. As described previously, SNV calling from each dataset was conducted [9]. The results of previous published analysis were advantageously used, demonstrating that the variant calling accuracy improves when relying on calls that can be established from both the forward or reverse strand (e.g. double-stranded) [9]. Of the 83 heterozygotes in high quality genomic DNA from flash-frozen tissue, 71 were also called from the FFPE-derived DNA for individual 751 (85%). Similar sensitivity values for the other two patients (84% and 85% respectively for individuals 761 and 780) were obtained.

### Example 3

### Evaluation of Sequencing Errors from the Archival Process

Given that matched samples from normal tissue of the same individual are used, differences between the SNV-calling results between FFPE versus flash-frozen derived DNA is attributable to FFPE-induced damage. Sequencing-related errors were eliminated based on the triplicate resequencing of each sample. As previously published, a straightforward statistical method to identify differences between matched samples which were previously applied to normal tumor pairs [9] was developed. At any given sequence position, the present method imposes that the difference in the second most frequent bases between the two samples exceeds 10% for both forward and reverse strand aligning reads. The 14 datasets were analyzed as seven matched pairs comparing sequence data from matched FFPE versus flash-frozen derived genomic DNA samples. The analysis yielded an average of 10.2 FFPE-specific calls (standard deviation being 4.2) per pair within the 102 Kb target (N=73 total positions for all pairs representing 45 unique positions). This results in one false positive call per every 12 Kb of targeted DNA. The FFPE-specific calls are replicated amongst the datasets that were sequenced in triplicate (patients 751 and 761) indicating that these errors were not attributable to the sequencing chemistry or processing but inherently found in the FFPE-derived DNA. There was no overlap between patients amongst these FFPE specific calls.

The pattern of FFPE-specific substitution errors were examined (Table 2). For substitutions, there are twelve combinations when considering all possibilities. Thirty one changes were transitions and 14 were transversions. Only 4 categories of substitutions among the 12 different substitutions were observed. This represented 44 out of the 45 observed cases. Nearly all of the observed changes obey the consensus G or C→A or T. The C→T and G→A transitions are compatible with cytosine deamination which is a common FFPE processing artifact [10].

TABLE 2
Substitutions specific to targeted resequencing of the FFPE sample

| | |
|---|---|
| Fresh | FFPE base |

| base | A | G | C | T |
|---|---|---|---|---|
| A | | 0 | 0 | 0 |
| G | 12 | | 0 | 8 |
| C | 6 | 0 | | 18 |
| T | 0 | 0 | 1 | |

Non-bolded: Transversions
Bolded: Transitions
Consensus: G or C → A or T

The above table shows that the chemical treatment involved in the FFPE process causes far fewer single base changes than are normally observed between individuals in the form of SNPs. Further, these chemical modifications are predictable as most likely being G->A or C->T. This means that the present methodology can be useful in an SNP analysis of genomic DNA from an FFPE sample.

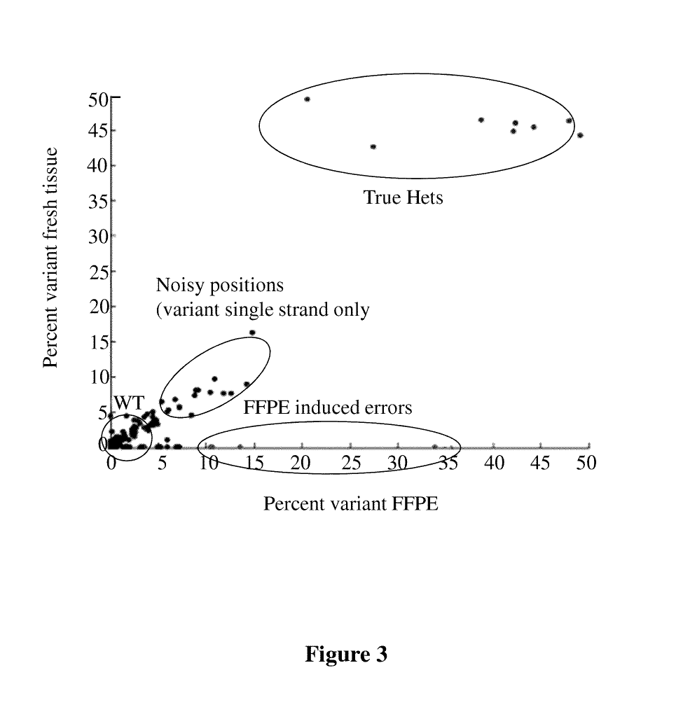

It is noted that while just one position per 12 kb of targeted sequence results in an FFPE specific calls that passed a statistical significance cutoff for significance and was found in both the forward and reverse strands of capture sequence. From either FFPE or flash-frozen derived genomic DNA, a number of positions had suggestions of a variant but were typically seen only the forward or the reverse strand. Using the variant calling method which imposes double-stranded representation, these positions were effectively eliminated as false positive calls (FIG. 3).

### Example 4

### Optimizing Capture Oligonucleotide Parameters

Having obtained promising results from the initial capture oligonucleotides, an improved bioinformatic pipeline for in silico capture oligonucleotide design was developed. The present design process optimizes the placement of the targeting arms according to the following considerations: (1) it attempts to place the 20 bp targeting arms in positions unique over the genome and that have no single mismatch neighbor, (2) identifying capture arms with GC content between 30% and 60%, (3) the size distribution of the target genomic regions approximating 220 bases in length. The new design process was applied to the targeting 80 exons from six cancer genes. A total of 288 capture oligonucleotides were synthesized for this six gene capture assay and these pooled oligonucleotides were used on three matched normal and tumors samples from the same individual. One DNA sample was obtained from flash-frozen tumor tissue, one sample was obtained from an FFPE section and a third normal DNA sample was obtained peripheral lymphocytes. Significantly improved performance metrics were noted using these optimized capture parameters.

Further optimization of the present process was carried out to show amplicon length obtained at different temperatures with the 628 capture oligonucleotides used. Ranges from 50 deg. to 60 deg. annealing temperatures showed no size bias between an amplicon length of 150-250 bp. Annealing temperature of 50 deg. was shown to yield a higher number of amplified targets. Also, consistent coverage across the amplicon lengths between 150 and 250 bp was shown. It was also shown that the process was tolerant of hairpin structures that can form in ssDNA that is being captured by the present capture probes.

As another novel feature, the sequencing library adapter sequences were incorporated into the universal vector sequence. This enabled a sequencing read library with a single amplification step to be generated, thus significantly reducing the complexity of the workflow used for next generation sequencing instruments such as the Illumina HiSeq, GAIIx, MiSeq, Life Sciences Solid, Ion Torrent, Pacific Biosciences system and the Roche 454 sequencer among others.

The present compositions may be provided in kit form, comprising a set of capture probes and universal oligonucleotides. Primers and a polymerase for amplification may also be included in the kit.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

### REFERENCES

1. Albert T J, Molla M N, Muzny D M, Nazareth L, Wheeler D, Song X, Richmond T A, Middle C M, Rodesch M J, Packard C J, et al: Direct selection of human genomic loci by microarray hybridization. Nat Methods 2007, 4:903-905.
2. Hodges E, Xuan Z, Balija V, Kramer M, Molla M N, Smith S W, Middle C M, Rodesch M J, Albert T J, Hannon G J, McCombie W R: Genome-wide in situ exon capture for selective resequencing. Nat Genet. 2007, 39:1522-1527.
3. Okou D T, Steinberg K M, Middle C, Cutler D J, Albert T J, Zwick M E: Microarray-based genomic selection for high-throughput resequencing. Nat Methods 2007, 4:907-909.
4. Gnirke A, Melnikov A, Maguire J, Rogov P, Leproust E, Brockman W, Fennell T, Giannoukos G, Fisher S, Russ C, et al: Solution hybrid selection with ultra-long oligonucleotides for massively parallel targeted sequencing. Nat Biotechnol 2009.
5. Varley K E, Mitra R D: Nested Patch PCR enables highly multiplexed mutation discovery in candidate genes. Genome Res 2008, 18:1844-1850.
6. Tewhey R, Warner J B, Nakano M, Libby B, Medkova M, David P H, Kotsopoulos S K, Samuels M L, Hutchison J B, Larson J W, et al: Microdroplet-based PCR enrichment for large-scale targeted sequencing. Nat Biotechnol 2009, 27:1025-1031.
7. Porreca G J, Zhang K, Li J B, Xie B, Austin D, Vas sallo SL, LeProust E M, Peck B J, Emig C J, Dahl F, et al: Multiplex amplification of large sets of human exons. Nat Methods 2007, 4:931-936.
8. Turner E H, Lee C, Ng S B, Nickerson D A, Shendure J: Massively parallel exon capture and library-free resequencing across 16 genomes. Nat Methods 2009, 6:315-316.
9. Natsoulis G, Bell J M, Xu H, Buenrostro J D, Ordonez H, Grimes S, Newburger D, Jensen M, Zahn J M, Zhang N, Ji H P: A flexible approach for highly multiplexed candidate gene targeted resequencing. PLOS one 2011, 6:e21088.
10. Kerick M, Isau M, Timmermann B, Sultmann H, Herwig R, Krobitsch S, Schaefer G, Verdorfer I, Bartsch G, Klocker H, et al: Targeted high throughput sequencing in clinical cancer Settings: formaldehyde fixed-paraffin embedded (FFPE) tumor tissues, input amount and tumor heterogeneity. BMC Med Genomics 2011, 4:68.
11. Ji H, Welch K: Molecular inversion probe assay for allelic quantitation. Methods Mol Biol 2009, 556:67-87.
12. Lehman I R, Nussbaum A L: The Deoxyribonucleases of Escherichia Coli. V. On the Specificity of Exonuclease I (Phosphodiesterase). J Biol Chem 1964, 239:2628-2636.
13. Flaherty P, Natsoulis G, Muralidharan O, Winters M, Buenrostro J, Bell J, Brown S, Holodniy M, Zhang N, Ji H P: Ultrasensitive detection of rare mutations using next-generation targeted resequencing. Nucleic Acids Res 2011.
14. Korn J M, Kuruvilla F G, McCarron S A, Wysoker A, Nemesh J, Cawley S, Hubbell E, Veitch J, Collins P J, Darvishi K, et al: Integrated genotype calling and association analysis of SNPs, common copy number polymorphisms and rare CNVs. Nat Genet. 2008, 40:1253-1260.
15. Lyamichev V, Brow M A, Dahlberg J E: Structure-specific endonucleolytic cleavage of nucleic acids by eubacterial DNA polymerases. Science 1993, 260:778-783.

MEGA

What is claimed is:

1. A composition useful for preparing a population of double stranded DNA molecules from a sample containing single stranded polynucleic acids, comprising:
(a) a plurality of polynucleotide capture probes, wherein individual capture probes each contain (i) capture arms at a 3′ end a 5′ end of the probe for hybridizing to specific portions of a single stranded polynucleic acid in the sample and (ii) an invariant sequence between the capture arms, whereby a circular structure comprising a specific capture probe and a polynucleic acid having regions complementary to the capture arms is formed;
(b) a plurality of second polynucleotides having a sequence complementary to the invariant sequence;
(c) a 5′ exonuclease;
(d) a 3′ exonuclease; and
(e) a ligase.

2. The composition of claim 1 further comprising at least one of (a) PCR amplification and (b) a DNA polymerase.

3. The composition of claim 1, further comprising a sample comprising single stranded polynucleic acids which are fragments of human genomic DNA.

4. The composition of claim 1, further comprising a sample comprising single stranded polynucleic acids which are fragments of human genomic DNA that have been fixed by crosslinking and embedded in a wax.

5. The composition of claim 1, wherein the polynucleotide capture probes in the composition comprise at least 500 different capture arm sequences.

6. The composition of claim 1, wherein the 5′ exonuclease is Exonuclease I.

7. The composition of claim 1, wherein the 3′ exonuclease is also a DNA polymerase.

8. The composition of claim 1, wherein the 3′ exonuclease is a thermostable DNA polymerase.

9. The composition of claim 1, wherein the ligase is a thermostable DNA ligase and the circular structure is formed by DNA molecules.

10. The composition of claim 1, wherein the second polynucleotide comprises PCR amplification sites and the composition comprises PCR primers complementary thereto.

11. The composition of claim 10, wherein the PCR amplification sites are spaced on the second polynucleotides about 120 to 250 bases apart.

12. A method for analyzing single stranded polynucleotides in a sample, comprising the steps of:
(a) adding to the sample a plurality of polynucleotide capture probes, each capture probe containing capture arms complementary to specific portions of a polynucleic acid in the sample and an invariant sequence between the arms, whereby a circular structure comprising a specific capture probe and a polynucleic acid sample molecule is formed;
(b) adding to the sample a plurality of second polynucleotides having a sequence complementary to the invariant sequence and having amplification sites for amplification of a polynucleic acid in a circular structure; and
(c) adding to the sample containing capture probes and second polynucleotides a mixture of a 5′ exonuclease, a 3′ exonuclease, and a ligase under conditions whereby exonucleases remove bases from the single stranded polynucleotides to form a new 5′ end thereof and a new 3′ end thereof, and the ligase ligates the new 5′ end to the new 3′ end.

13. The method of claim 12 further comprising the step of composition of claim 1 further comprising at least one of amplification primers and a polymerase.

14. The method of claim 12, wherein the single stranded polynucleic acids are fragments of human genomic DNA.

15. The method of claim 12, wherein the single stranded polynucleic acids are fragments of human genomic DNA that have been fixed by crosslinking and embedded in a wax.

16. The method of claim 12 wherein the capture probes comprise at least 500 different probes.

17. The method of claim 12, wherein the 5′ exonuclease is Exonuclease I.

18. The method of claim 12, wherein the 3′ exonuclease is also a polymerase.

19. The method of claim 12, wherein the 3′ exonuclease is a thermostable polymerase.

20. The method of claim 12, wherein the ligase is a thermostable DNA ligase and the circular structure is formed by DNA molecules.

21. A method for analyzing single stranded polynucleotides from a sample, comprising the steps of:
(a) adding to the sample a plurality of polynucleotide capture probes, each capture probe containing capture arms complementary to specific portions of a polynucleic acid in the sample and an invariant sequence between the arms, whereby a circular structure comprising a specific capture probe and a polynucleic acid sample molecule is formed in the buffer;
(b) adding to the sample a plurality of second polynucleotides having a sequence complementary to the invariant sequence and having amplification sites for amplification of a polynucleic acid in a circular structure; and
(c) adding to the sample containing capture probes and second polynucleotides a mixture of a 5′ exonuclease, a 3′ exonuclease, and a ligase under conditions whereby exonucleases remove bases from the single stranded polynucleotides to form a new 5′ end thereof and a new 3′ end thereof, and the ligase ligates the new 5′ end to the new 3′ end;
(d) adding to the sample a polymerase and polymerase primers; and
(e) conducting a polymerase chain reaction using the polymerase primers for amplification of a portion of a single stranded polynucleotide captured by a corresponding capture probe.

22. The method of claim 21 further comprising the step of sequencing amplified polynucleotides from step (e).

23. The method of claim 21 wherein the polymerase chain reaction utilizes an annealing temperature of between about 45 degrees Celsius and 55 degrees Celsius.

24. The method of claim 21 wherein the analyzing single stranded polynucleotides from a sample comprises analyzing polynucleotides from a preserved tissue sample or analyzing polynucleotides from a preserved tissue sample and analyzing polynucleotides from a fresh sample from the same individual.

25. A kit for preparing a composition according to claim 1 comprising:
(a) a plurality of capture probes, each capture probe containing (i) 5′ and 3′ end capture arms complementary to specific portions of a polynucleic acid in the sample and (ii) an invariant sequence between the capture arms, whereby a circular structure comprising a specific capture probe and a polynucleic acid sample molecule having regions complementary to the capture arms is formed in the buffer;
(b) a plurality of second polynucleotides having a sequence complementary to the invariant sequence and having amplification sites for amplification of a polynucleic acid in a circular structure; and
(c) a 5′ exonuclease, a 3′ exonuclease, and a ligase.

26. The kit according to claim 25, wherein said kit further comprises at least one of amplification primers and a polymerase.
